SEQUENCE LISTING (18 sequences)

SEQ ID NO: 1 - DNA, 1486 bp, Brevibacillus parabrevis NITE BP-03020
cgaacgctggcggcgtgcctaatacatgcaagtcgagcgagggttttcggaccctagcggcggacgggtgagtaacacgtaggcaacctgcctctcagaccgggataacatagggaaacttatgctaataccggataggtttttggattgcatgatccgaaaagaaaagatggcttcggctatcactgggagatgggcctgcggcgcattagctagttggtggggtaacggcctaccaaggcgacgatgcgtagccgacctgagagggtgaccggccacactgggactgagacacggcccagactcctacgggaggcagcagtagggaattttccacaatggacgaaagtctgatggagcaacgccgcgtgaacgatgaaggtcttcggattgtaaagttctgttgtcagggacgaacacgtrccgttcgaatagggcggyaccttgacggtacctgacgagaaagccacggctaactacgtgccagcagccgcggtaatacgtaggtggcaagcgttgtccggatttattgggcgtaaagcgcgcgcaggcggctatgtaagtctggtgttaaagcccggagctcaactccggttcgcatcggaaactgtgtagcttgagtgcagaagaggaaagcggtattccacgtgtagcggtgaaatgcgtagagatgtggaggaacaccagtggcgaaggcggctttctggtctgtaactgacgctgaggcgcgaaagcgtggggagcaaacaggattagataccctggtagtccacgccgtaaacgatgagtgctaggtgttgggggtttcaataccctcagtgccgcagctaacgcaataagcactccgcctggggagtacgctcgcaagagtgaaactcaaaggaattgacgggggcccgcacaagcggtggagcatgtggtttaattcgaagcaacgcgaagaaccttaccaggtcttgacatcccgctgaccgctctggagacagagcttcccttcggggcagcggtgacaggtggtgcatggttgtcgtcagctcgtgtcgtgagatgttgggttaagtcccgcaacgagcgcaacccttatctttagttgccagcattcagttgggcactctagagagactgccgtcgacaagacggaggaaggcggggatgacgtcaaatcatcatgccccttatgacctgggctacacacgtgctacaatggttggtacaacgggatgctacctcgcgagrggacgccaatctctgaaaaccaatctcagttcggattgtaggctgcaactcgcctacatgaagtcggaatcgctagtaatcgcggatcagcatgccgcggtgaatacgttcccgggccttgtacacaccgcccgtcacaccacgggagtttgcaacacccgaagtcggtgaggtaaccgcaaggagccagccgccgaaggtggggtagatgactggggtgaagtcgtaacaaggtatccgtaccggaagg

SEQ ID NO: 2 - DNA, 20 bp, Artificial Sequence
  synthetic
agrgtttgatcmtggctcag

SEQ ID NO: 3 - DNA, 19 bp, Artificial Sequence
  synthetic
ggytaccttgttacgactt

SEQ ID NO: 4 - DNA, 19 bp, Artificial Sequence
  synthetic
ctcctacgggaggcagcag

SEQ ID NO: 5 - DNA, 18 bp, Artificial Sequence
  synthetic
gtattaccgcggctgctg

SEQ ID NO: 6 - DNA, 20 bp, Artificial Sequence
  synthetic
aaactyaaaggaattgacgg

SEQ ID NO: 7 - DNA, 1471 bp, Tumebacillus sp. NITE BP-02779
acgaacgctggcggcgtgcctaatacatgcaagtcgagcggagtgtmgagagcttgctctcwraggtcagcggcggacgggtgagtaacacgtgggtaacctgcctggcagactgggataacgcttggaaacgagtgctaataccggataatctcttggatcgcatggtctgagagtaaaaggagcttttgcttcactgcyagatggacccgcggcgcattagctagttggtgaggtaatggctcaccaaggcgacgatgcgtagccgacctgagagggtgaccggccacactgggactgagacacggcccagactcctacgggaggcagcagtagggaatcttccgcaatgggcgcaagcctgacggagcaacgccgcgtgagtgatgaaggccttcgggttgtaaaactctgtcttctgtgaagaaccatcctgtgcagagaaagctcaggacctgacggtaacagaggaggaagccccggctaactacgtgccagcagccgcggtaatacgtagggggcaagcgttgtccggaatcactgggcgtaaagcgcgcgcaggcggtctctcacgtccggggtgaaagcccagagctcaactctgggattgccttggatacggggagacttgaggatcggagaggcaaggggaattccacgtgtagcggtgaaatgcgtagagatgtggaggaacacctgtggcgaaggcgccttgctggccgatttctgacgctgaggcgcgaaagcgtggggagcaaacaggattagataccctggtagtccacgccgtaaacgatgagtgctaggtgttagggggcccaccccttagtgccgaagctaacgcattaagcactccgcctggggagtacggtcgcaagactgaaactcaaaggaattgacgggggcccgcacaagcagtggagcatgtggtttaattcgaagcaacgcgaagaaccttaccaagacttgacatcccgctgaccrctctagagatagagyttcccttcggggcagcggtgacaggtggtgcatggttgtcgtcagctcgtgtcgtgagatgttgggttaagtcccgcaacgagcgcaacccctatgttgtgttgctaccatttagttgagcacttacaacagactgccggtgacaaaccggaggaaggcggggatgacgtcaaatcatcatgccccttatgtcttgggctacacacgtgctacaatggacggtacaaagggttgcgaagccgcgaggtgaagccaatcccagaaagccgttcgtagttcggattgcaggctgcaactcgcctgcatgaagccggaattgctagtaatcgcggatcagcatgccgcggtgaattcgttcccgggccttgtacacaccgcccgtcacaccatgggagttggtaacacccgaagtcggtgaggtaactgcttgcagagccagccgcctaaggtgggatcgataactggggtgaag

SEQ ID NO: 8 - DNA, 1538 bp, B.subtilis subsp.inaquosorum DSM022148
tttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatgcttgtttgaaccgcatggttcaaacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtcgtaacaaggtagccgtatcggaaggtgcggctggatcacctcctt

SEQ ID NO: 9 - DNA, 1470 bp, Tumebacillus algifaecis NBRC108765t
  misc_feature (45)..(45): n is a, c, g, or t
  misc_feature (159)..(159): n is a, c, g, or t
  misc_feature (161)..(161): n is a, c, g, or t
  misc_feature (172)..(173): n is a, c, g, or t
  misc_feature (443)..(443): n is a, c, g, or t
  misc_feature (1427)..(1427): n is a, c, g, or t
gacgaacgctggcggcgtgcctaatacatgcaagtcgagcggacnggagggagcttgctcccaaaggttagcggcggacgggtgagtaacacgtgggcaatctgcccgacagactgggataacgcttggaaacgagtgctaataccggataagcgattncntcgcatgaggnnatcgagaaagaagctttcgcttcactgtcggatgagcccgcggcgcattagctagttggtgaggtaacggctcaccaaggcgacgatgcgtagccgacctgagagggtgatcggccacactgggactgagacacggcccagactcctacgggaggcagcagtagggaatcttccacaatgggcgcaagcctgatggagcaacgccgcgtgaatgatgaaggccttcgggttgtaaaattctgtcttctgtgaagaacaagtgtgagaagngaatgctcacaccctgacggtaacagaggaggaagccccggctaactacgtgccagcagccgcggtaatacgtagggggcaagcgttgtccggaatcactgggcgtaaagcgcgcgcaggcggccatctgcgtccggggtgaaagcccaaggctcaaccttgggactgccttggatacgggatggcttgaggatcggagaggcaaggggaattccacgtgtagcggtgaaatgcgtagagatgtggaggaacacctgtggcgaaggcgccttgctggccgatttctgacgctgaggcgcgaaagcgtggggagcaaacaggattagataccctggtagtccacgccgtaaacgatgagtgctaggtgttggggggtaccaccctcagtgccgaagctaacgcattaagcactccgcctggggagtacggtcgcaagactgaaactcaaaggaattgacgggggcccgcacaagcagtggagcatgtggtttaattcgaagcaacgcgaagaaccttaccaagacttgacatcccgctgaccggtttagagatagaccttcccttcggggcagcggtgacaggtggtgcatggttgtcgtcagctcgtgtcgtgagatgttgggttaagtcccgcaacgagcgcaacccctaaattgtgttgccatcattcagttgggcactcacaattgactgccggtgacaaaccggaggaaggcggggatgacgtcaaatcatcatgccccttatgtcttgggctacacacgtgctacaatgggcggtacaaagggttgcgaggccgcgaggcggagccaatcccaaaaagccgctcacagttcggattgcaggctgcaactcgcctgcatgaagctggaattgctagtaatcgcggatcagcatgccgcggtgaattcgttcccgggccttgtacacaccgcccgtcacaccatgggagttggcaacacccgaagccggtgaggtaaccgnaaggagccagccgtctaaggtggggtcgatgactggggtgaag

SEQ ID NO: 10 - DNA, 1479 bp, Paenibacillus polymyxa JCM 2507t
  misc_feature (1004)..(1004): n is a, c, g, or t
  misc_feature (1248)..(1249): n is a, c, g, or t
  misc_feature (1254)..(1255): n is a, c, g, or t
gacgaacgctggcggcgtgcctaatacatgcaagtcgagcggggttatttagaagcttgcttctaattaacctagcggcggacgggtgagtaacacgtaggcaacctgcccacaagacagggataactaccggaaacggtagctaatacccgatacatccttttcctgcatgggagaaggaggaaaggcggagcaatctgtcacttgtggatgggcctgcggcgcattagctagttggtggggtaaaggcctaccaaggcgacgatgcgtagccgacctgagagggtgatcggccacactgggactgagacacggcccagactcctacgggaggcagcagtagggaatcttccgcaatgggcgaaagcctgacggagcaacgccgcgtgagtgatgaaggttttcggatcgtaaagctctgttgccagggaagaacgtcttgtagagtaactgctacaagagtgacggtacctgagaagaaagccccggctaactacgtgccagcagccgcggtaatacgtagggggcaagcgttgtccggaattattgggcgtaaagcgcgcgcaggcggctctttaagtctggtgtttaatcccgaggctcaacttcgggtcgcactggaaactggggagcttgagtgcagaagaggagagtggaattccacgtgtagcggtgaaatgcgtagagatgtggaggaacaccagtggcgaaggcgactctctgggctgtaactgacgctgaggcgcgaaagcgtggggagcaaacaggattagataccctggtagtccacgccgtaaacgatgaatgctaggtgttaggggtttcgatacccttggtgccgaagttaacacattaagcattccgcctggggagtacggtcgcaagactgaaactcaaaggaattgacggggacccgcacaagcagtggagtatgtggtttaattcgaagcaacgcgaagaaccttaccaggtcttgacatccctctgaccggtctagagatagncctttccttcgggacagaggagacaggtggtgcatggttgtcgtcagctcgtgtcgtgagatgttgggttaagtcccgcaacgagcgcaacccttatgcttagttgccagcaggtcaagctgggcactctaagcagactgccggtgacaaaccggaggaaggtggggatgacgtcaaatcatcatgccccttatgacctgggctacacacgtactacaatggccggtacaacgggaagcgaagnngcganntggagccaatcctagaaaagccggtctcagttcggattgtaggctgcaactcgcctacatgaagtcggaattgctagtaatcgcggatcagcatgccgcggtgaatacgttcccgggtcttgtacacaccgcccgtcacaccacgagagtttacaacacccgaagtcggtgaggtaaccgcaaggagccagccgccgaaggtggggtagatgattggggtgaag

SEQ ID NO: 11 - DNA, 1475 bp, Bacillus amyloliquefaciens NBRC3037
  misc_feature (152)..(152): n is a, c, g, or t
gacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatgnttgtttgaaccgcatggttcagacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttatggagccagccgccgaaggtgggacagatgattggggtgaag

SEQ ID NO: 12 - DNA, 1475 bp, Bacillus subtilis IFO 3134
gacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatgcttgtttgaaccgcatggttcaaacataaaaggtggcttcggctaccacttacagatggacccgcggcgcattagctagttggtgaggtaatggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggttccttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaag

SEQ ID NO: 13 - DNA, 1475 bp, Bacillus subtilis IFO 3026
gacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaag

SEQ ID NO: 14 - DNA, 1475 bp, Bacillus subtilis IFO 13169
  misc_feature (257)..(257): n is a, c, g, or t
gacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaacggctcaccaaggcnacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaag

SEQ ID NO: 15 - DNA, 1478 bp, Bacillus sphaericus IFO 3341
gacgaacgctggcggcgtgcctaatacatgcaagtcgagcgaacagaaaaggagcttgctcctttgacgttagcggcggacgggtgagtaacacgtgggcaacctaccctatagtttgggataactccgggaaaccggggctaataccgaataatctcttttacttcatggtgaaagactgaaagacggcatctcgctgtcgctataggatgggcccgcggcgcattagctagttggtgaggtaacggctcaccaaggcgacgatgcgtagccgacctgagagggtgatcggccacactgggactgagacacggcccagactcctacgggaggcagcagtagggaatcttccacaatgggcgaaagcctgatggagcaacgccgcgtgagtgaagaaggttttcggatcgtaaaactctgttgtaagggaagaacaagtacagtagtaactggctgtaccttgacggtaccttattagaaagccacggctaactacgtgccagcagccgcggtaatacgtaggtggcaagcgttgtccggaattattgggcgtaaagcgcgcgcaggcggtcctttaagtctgatgtgaaagcccacggctcaaccgtggagggtcattggaaactgggggacttgagtgcagaagaggaaagtggaattccaagtgtagcggtgaaatgcgtagagatttggaggaacaccagtggcgaaggcgactttctggtctgtaactgacgctgaggcgcgaaagcgtggggagcaaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcccgttgaccactgtagagatatagtttccccttcgggggcaacggtgacaggtggtgcatggttgtcgtcagctcgtgtcgtgagatgttgggttaagtcccgcaacgagcgcaacccttgatcttagttgccatcatttagttgggcactctaaggtgactgccggtgacaaaccggaggaaggtggggatgacgtcaaatcatcatgccccttatgacctgggctacacacgtgctacaatggacgatacaaacggttgccaactcgcgagagggagctaatccgataaagtcgttctcagttcggattgtaggctgcaactcgcctacatgaagccggaatcgctagtaatcgcggatcagcatgccgcggtgaatacgttcccgggccttgtacacaccgcccgtcacaccacgagagtttgtaacacccgaagtcggtgaggtaaccttttggagccagccgccgaaggtgggatagatgattggggtgaag

SEQ ID NO: 16 - DNA, 1477 bp, Bacillus sphaericus IFO 3528
gacgaacgctggcggcgtgcctaatacatgcaagtcgagcgaacagagaaggagcttgctccttcgacgttagcggcggacgggtgagtaacacgtgggcaacctaccttatagtttgggataactccgggaaaccggggctaataccgaataatctgtttcacctcatggtgaaacactgaaagacggtttcggctgtcgctataggatgggcccgcggcgcattagctagttggtgaggtaacggctcaccaaggcgacgatgcgtagccgacctgagagggtgatcggccacactgggactgagacacggcccagactcctacgggaggcagcagtagggaatcttccacaatgggcgaaagcctgatggagcaacgccgcgtgagtgaagaaggatttcggttcgtaaaactctgttgtaagggaagaacaagtacagtagtaactggctgtaccttgacggtaccttattagaaagccacggctaactacgtgccagcagccgcggtaatacgtaggtggcaagcgttgtccggaattattgggcgtaaagcgcgcgcaggtggtttcttaagtctgatgtgaaagcccacggctcaaccgtggagggtcattggaaactgggagacttgagtgcagaagaggatagtggaattccaagtgtagcggtgaaatgcgtagagatttggaggaacaccagtggcgaaggcgactatctggtctgtaactgacactgaggcgcgaaagcgtggggagcaaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcccgttgaccactgtagagatatagtttccccttcgggggcaacggtgacaggtggtgcatggttgtcgtcagctcgtgtcgtgagatgttgggttaagtcccgcaacgagcgcaacccttgatcttagttgccatcatttagttgggcactctaaggtgactgccggtgacaaaccggaggaaggtggggatgacgtcaaatcatcatgccccttatgacctgggctacacacgtgctacaatggacgatacaaacggttgccaactcgcgagagggagctaatccgataaagtcgttctcagttcggattgtaggctgcaactcgcctacatgaagccggaatcgctagtaatcgcggatcagcatgccgcggtgaatacgttcccgggccttgtacacaccgcccgtcacaccacgagagtttgtaacacccgaagtcggtgaggtaaccttttggagccagccgccgaaggtgggatagatgattggggtgaag

SEQ ID NO: 17 - DNA, 1475 bp, Bacillus licheniformis IFO 12197
gacgaacgctggcggcgtgcctaatacatgcaagtcgagcggacagatgggagcttgctccctgatgtcagcggcggacgggtgagtaacacgtgggtaacctgcctgtaagactgggataactccgggaaaccggggctaataccggatgcttgattgaaccgcatggttcaattataaaaggtggcttttagctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaaactctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagcgcgcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggcgcgaaagcgtggggagcgaacaggattagataccctggtagtccacgccgtaaacgatgagtgctaagtgttagagggtttccgccctttagtgctgcagcaaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggcttccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatgggcagaacaaagggcagcgaagccgcgaggctaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttggagccagccgccgaaggtgggacagatgattggggtgaag

SEQ ID NO: 18 - DNA, 1475 bp, Bacillus subtilis 168 NBRC11470
  misc_feature (437)..(437): n is a, c, g, or t
gacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtnccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaag

(19) United States
(12) Patent Application Publication
(10) Pub. No.: US 2023/0042056 A1
(43) Pub. Date: Feb. 9, 2023

(54) BACTERIUM FOR DEGRADING SLUDGE, BACTERIUM DEGRADING MICROORGANISM, MICROBIAL PREPARATION AND METHOD AND DEVICE FOR DEGRADING SLUDGE

(71) Applicants: SUMITOMO CHEMICAL COMPANY, LIMITED, Tokyo (JP); KATAOKA BIO LABORATORY CO., LTD., Isehara-shi, Kanagawa (JP)

(72) Inventor: Shinya HIRAYAMA, Osaka-shi, Osaka (JP)

(73) Assignees: SUMITOMO CHEMICAL COMPANY, LIMITED, Tokyo (JP); KATAOKA BIO LABORATORY CO., LTD., Isehara-shi, Kanagawa (JP)

(21) Appl. No.: 17/788,427

(22) PCT Filed: Dec. 17, 2020
(86) PCT No.: PCT/JP2020/047273

§ 371 Date: Jun. 23, 2022

(30) Foreign Application Priority Data
Dec. 24, 2019 (JP) 2019-233401

(51) Int. Cl. C02F 11/02; C12N 1/20; C02F 3/34

(52) U.S. Cl. CPC C02F 11/02; C12N 1/205; C02F 3/34; C12R 2001/01

(57) ABSTRACT

Provided are a bacterium for degrading sludge having a 16S rRNA gene comprising a nucleotide sequence having 97% or more identity to the nucleotide sequence represented by SEQ ID NO: 1, a bacterium having a 16S rRNA gene comprising the nucleotide sequence represented by SEQ ID NO: 1 with mutations of two bases or less, and having an ability to degrade a target microorganism, and a microbial preparation for degrading a target microorganism comprising a bacterium (a1) below. Bacterium (a1) is a bacterium having a 16S rRNA gene comprising a nucleotide sequence having 90% or more identity to the nucleotide sequence represented by SEQ ID NO: 1.

### TECHNICAL FIELD

The present invention relates to bacteria for degrading sludge, bacterium degrading microorganisms, microbial preparations, and methods and devices for degrading sludge.

### BACKGROUND ART

When waste water is decontaminated by an activated sludge process, sludge called excess sludge, which contains microorganisms grown by degrading organic matters, is generated. Excess sludge that is discharged from waste water treatment plants accounts for 40% or more of the industrial wastes, and it is thus considered effective to reduce a volume of such excess sludges to reduce a volume of the industrial wastes. Excess sludge is generally dewatered, dried, and then incineration treated (NPL 1: “Fiscal year of Heisei 27, Report on current status of industrial waste treatment, Results of Heisei 25 (Digest edition),” [online], March, Heisei 28, Minister's Secretariat, Ministry of the Environment Waste Management and Recycling Department, [searched on August 31, Heisei 30], Internet <URL:https://www.e-stat.go.jp/stat-search/file-download?statInfId=000031403476&fileKind=2>; NPL 2: YAMAMOTO Masayuki, “Combustion Technology of the Sewage”, Journal of the Combustion Society of Japan, Combustion Society of Japan, 2011, Vol. 53, 164, p91-96).

However, incineration treatment of excess sludge generates greenhouse gasses, and thus such a treatment was not necessarily a treatment method with little impact on the environment.

### CITATION LIST

### Non Patent Literature

NPL 1: “Fiscal year of Heisei 27, Report on current status of industrial waste treatment, Results of Heisei 25 (Digest edition),” [online], March, Heisei 28, Minister's Secretariat, Ministry of the Environment Waste Management and Recycling Department, [searched on August 31, Heisei 30], Internet <URL:https://www.e-stat.go.jp/stat-search/file-download? statInfId=000031403476&fileKind=2>
NPL 2: YAMAMOTO Masayuki, “Combustion Technology of the Sewage”, Journal of the Combustion Society of Japan, Combustion Society of Japan, 2011, Vol. 53, 164, p91-96).

### SUMMARY OF INVENTION

### Technical Problem

In recent years, the society as a whole is having an increasing awareness of the global environment, and there has been a demand for a method of reducing a volume of excess sludge with even lesser impact imposed on the environment, that is, a method for degrading microorganisms composing excess sludge.

The present invention has an object to provide bacteria for degrading sludge, a bacterium degrading microorganism, a microbial preparation, a method and a device for degrading sludge.

### Solution to Problem

That is, the present invention relates to [1] to [18] given below.

[1] A bacterium for degrading sludge having a 16S rRNA gene comprising a nucleotide sequence having 97% or more identity to the nucleotide sequence represented by SEQ ID NO: 1 (hereinafter, also referred to as “the bacteria for degrading sludge according to the present invention”).
[2] A bacterium having a 16S rRNA gene comprising the nucleotide sequence represented by SEQ ID NO: 1 with mutations of two bases or less, and having an ability to degrade a target microorganism (hereinafter, also referred to as “the bacteria according to the present invention”).
[3] The bacterium according to [2], wherein the target microorganism is a target bacterium.
[4] The bacterium according to [2] or [3], wherein the target microorganism is a target killed bacterium.
[5] The bacterium according to any of [1] to [4], having a 16S rRNA gene comprising the nucleotide sequence represented by SEQ ID NO: 1.
[6] A bacterium deposited under Accession number NITE BP-03020.
[7] A microbial preparation for degrading sludge, comprising a bacterium (a1): (hereinafter, also referred to as “the microbial preparation according to the present invention”)

bacterium (a1): A bacterium having a 16S rRNA gene comprising a nucleotide sequence having 90% or more identity to the nucleotide sequence represented by SEQ ID NO: 1.

[8] The microbial preparation for degrading sludge according to [7], wherein the bacterium (a1) has a 16S rRNA gene comprising a nucleotide sequence having 95% or more identity to the nucleotide sequence represented by SEQ ID NO: 1.
[9] The microbial preparation for degrading sludge according to [7], wherein the bacterium (a1) has a 16S rRNA gene comprising a nucleotide sequence having 97% or more identity to the nucleotide sequence represented by SEQ ID NO: 1.
[10] The microbial preparation for degrading sludge according to [7], wherein the bacterium (a1) has a 16S rRNA gene comprising the nucleotide sequence represented by SEQ ID NO: 1.
[11] The microbial preparation for degrading sludge according to any one of [7] to [10], wherein the bacterium (a1) has an ability to degrade a target microorganism.
[12] The microbial preparation for degrading sludge according to any one of [7] to [11], which degrades sludge at a temperature of 20° C.
[13] The microbial preparation for degrading sludge according to [7], wherein the bacterium (a1) is a bacterium deposited under Accession number NITE BP-03020.
[14] The microbial preparation for degrading sludge according to any one of [7] to [13], further comprising a microorganism (a2):

microorganism (a2): a microorganism different from bacterium (a1), wherein the percentage of the bacteria (a1) based on the total number of the bacteria (a1) and the microorganism (a2) is 0.1% or more and less than 100%.

[15] The microbial preparation for degrading sludge according to [14], wherein the microorganism (a2) has a 16S rRNA gene comprising a nucleotide sequence having 90% or more identity to the nucleotide sequence represented by SEQ ID NO: 7, and comprises at least one species selected from the group consisting of bacteria having an ability to degrade a target microorganism, bacteria of the genus Bacillus having an ability to degrade a target microorganism, and bacteria of the genus Paenibacillus having an ability to degrade a target microorganism.
[16] A microbial preparation for degrading sludge, comprising a culture of a bacterium (a1):

bacterium (a1): a bacterium having a 16S rRNA gene comprising a nucleotide sequence having 90% or more identity to the nucleotide sequence represented by SEQ ID NO: 1, and having an ability to degrade a target microorganism.

[17] A method for degrading sludge, comprising allowing the bacterium according to any one of [1] to [6] or the microbial preparation for degrading sludge according to any one of [7] to [16] to act on sludge.
[18] A device for degrading sludge, wherein the bacterium according to any of [1] to [6] or the microbial preparation for degrading sludge according to any of [7] to [16] is used (hereinafter, also referred to as “the degradation device according to the present invention”).

### Advantageous Effects of Invention

According to the present invention, bacteria for degrading sludge, a bacterium degrading microorganism, a microbial preparation, a method and a device for degrading microorganisms can be provided.

### BRIEF DESCRIPTION OF DRAWINGS

FIG. 1 is a photograph showing a shape of Brevibacillus parabrevis (hereinafter, also referred to as “Bb. parabrevis”) NITE BP-03020 in Experiment 3.

FIG. 2 is a photograph showing a result of Gram staining of Bb. parabrevis NITE BP-03020 in Experiment 3.

FIG. 3 is a graph showing an ability to degrade target microorganisms of Bb. parabrevis NITE BP-03020 in Experiment 4.

FIG. 4 is a graph showing an ability to degrade target microorganisms of Paenibacillus glycanilyticus in Experiment 4.

FIG. 5 is a graph showing an ability to degrade target microorganisms of microbial preparations containing a culture of multiple strains of Bb. parabrevis in Experiment 5.

FIG. 6 is a graph showing an ability to degrade a target microorganism (Micrococcus lysodeikticus) of the microbial preparation containing a culture of Bb. parabrevis NITE BP-03020 in Experiment 6.

FIG. 7 is a graph showing an ability to degrade a target microorganism (Saccharomyces cerevisiae) of the microbial preparation containing a culture of Bb. parabrevis NITE BP-03020 in Experiment 6.

FIG. 8 is a graph showing an ability to degrade a target microorganism (Escherichia coli) of the microbial preparation containing a culture of Bb. parabrevis NITE BP-03020 in Experiment 6.

FIG. 9 is a graph showing an ability to degrade a target microorganism (Brevibacillus parabrevis) of the microbial preparation containing a culture of Bb. parabrevis NITE BP-03020 in Experiment 6.

FIG. 10 is a graph showing an ability to degrade a target microorganism (Gluconobacter oxydans) of the microbial preparation containing a culture of Bb. parabrevis NITE BP-03020 in Experiment 6.

FIG. 11 is a graph showing an ability to degrade sludge of the microbial preparation containing a culture of Bb. parabrevis NITE BP-03020 in Experiment 7.

FIG. 12 is a graph showing an ability to degrade a target microorganism of the microbial preparation containing a culture of Bb. parabrevis NITE BP-03020 under various temperature conditions in Experiment 8.

FIG. 13 is a graph showing an ability to degrade sludge of the microbial preparation containing a mixture of a culture of Bb. parabrevis NITE BP-03020 and a culture of other bacteria in Experiment 9.

FIG. 14 is a chart showing a dry weight of excess sludge after addition of Tumebacillus sp. (NITE BP-02779) in Reference Experiment 7.

### DESCRIPTION OF EMBODIMENTS

Hereinafter, embodiments to carry out the present invention are described in detail. The present invention is not limited to the following embodiments.

(Descriptions of Common Terms)

In the present description, the “microorganism” is a microscopic organism whose structure is unidentifiable with the naked eye and is the organisms that exclude large multicellular organisms. The “target microorganisms” are microorganisms that are degraded by the bacteria or the microbial preparation according to the present invention. Examples of such target microorganisms include bacteria and fungi, and of which bacteria are preferable, with bacteria composing excess sludge being more preferable. The target microorganism can be a viable bacterium or a killed bacterium. The “target bacteria” refers to bacteria that are to be target microorganisms. The target bacteria can be viable bacteria or killed bacteria, or can include viable bacteria and killed bacteria. The viable bacterium refers to a live bacterium such as a bacterium in which the metabolism occurs. The killed bacteria refer to killed bacteria such as a bacterium in which the metabolism does not occur. A viable bacterium and a killed bacterium can be identified by using a stain such as propidium iodide (PI). The “target killed bacteria” refers to killed bacteria in the target bacteria. The target bacteria are preferably target killed bacteria from a viewpoint of the degradation efficiency. About 50% of the microorganisms in excess sludge is killed bacteria. Target killed bacteria can also be obtained by, for example, heating, autoclaving, UV irradiating, formalin-treating, or acid treating the target bacteria. The target killed bacteria can also be crushed bacteria.

Examples of the target microorganisms include gram-positive bacteria such as bacteria of the genus Micrococcus, bacteria of the genus Bacillus, bacteria of the genus Staphylococcus, bacteria of the genus Paenibacillus, and bacteria of the genus Lactobacillus, and gram-negative bacteria such as bacteria of the genus Escherichia, and bacteria of the genus Acetobacter, bacteria of the genus Gluconobacter, and fungi such as Saccharomyces cerevisiae.

In the present description, the “ability to degrade target microorganisms” refers to the ability to metabolize target microorganisms and convert biomolecules, a part or a whole, that compose the target microorganisms to different molecules. Examples of the biomolecule include sugars, proteins, nucleic acids, and lipids.

(Bacteria for Degrading Sludge According to the Present Invention)

The bacteria for degrading sludge according to the present invention has a 16S rRNA gene comprising a nucleotide sequence having 97% or more identity to the nucleotide sequence represented by SEQ ID NO: 1. Examples of the bacteria having a 16S rRNA gene comprising a nucleotide sequence having 97% or more identity to the nucleotide sequence represented by SEQ ID NO: 1 include bacteria of the genus Brevibacillus. Examples of bacteria of the genus Brevibacillus include Brevibacillus parabrevis, Brevibacillus choshinensis, Brevibacillus formosus, Brevibacillus agri, Brevibacillus nitrificans, Brevibacillus brevis, Brevibacillus reuszeri, Brevibacillus limnophilus, Brevibacillus panacihumi, and Brevibacillus gelatini.

The bacteria for degrading sludge according to the present invention, when compared with the nucleotide sequence represented by SEQ ID NO: 1, can have a 16S rRNA gene comprising a nucleotide sequence having 97.5% or more, 98% or more, 98.5% or more, 98.8% or more, 99.0% or more, 99.3% or more, 99.5% or more, 99.8% or more, or 99.9% or more identity thereto or homology therewith. The bacteria for degrading sludge can be a bacterium having a 16S rRNA gene comprising the nucleotide sequence represented by SEQ ID NO: 1, and examples of the representative strain of such bacteria include Brevibacillus parabrevis (Accession number NITE BP-03020) to be described later. The bacteria for degrading sludge according to the present invention can include the bacteria according to the present invention.

The bacteria for degrading sludge according to the present invention, when compared with the nucleotide sequence represented by SEQ ID NO: 1, can have a 16S rRNA gene comprising a nucleotide sequence in which one or several bases are substituted, deleted or added. One or several bases can be, for example, 1 to 25 bases, 1 to 10 bases, and 1 to 5 bases. The above mutation is a mutation by which the expression and functions of the 16S rRNA are not lost.

The bacteria for degrading sludge according to the present invention can have a 16S rRNA gene comprising a continuous sequence of about 15 bases or more, preferably about 18 to about 500 bases, more preferably about 18 to about 200 bases, and further preferably about 18 to about 50 bases, that are included in the nucleotide sequence represented by SEQ ID NO: 1, or a nucleotide sequence hybridizable to a complementary sequence thereof under stringent conditions. The stringent conditions refer to conditions under which a non-specific hybrid is not formed and examples include a condition under which one or more washings are carried out at 60° C. with 1× SSC in 0.1% SDS, and preferably at 68° C. with 0.1×SSC in 0.1% SDS.

The bacteria for degrading sludge according to the present invention has an ability to degrade sludge. Sludge is preferably excess sludge. The ability to degrade sludge varies depending on reaction conditions (kind and concentration of sludge and target microorganisms contained in the sludge, and composition, temperature, pH, number of bacteria of a solution containing bacteria for degrading sludge). Examples of the method for investigating the presence of ability to degrade sludge include a method in which sludge and a bacterium whose ability to degrade sludge needs to be investigated are reacted for a certain time in suitable medium or a buffer solution and then the degradation of the sludge in the medium or buffer solution is investigated. The method for investigating the degradation is not particularly limited and examples include a method of measuring a turbidity of sludge, a method of detecting target microorganisms contained in sludge using an SLP reagent, a method of detecting the DNA of target microorganisms contained in sludge by PCR, a method of measuring a dry weight of sludge, a method of measuring a dry weight of target microorganisms contained in sludge, and a method of detecting degraded products derived from target microorganisms contained in sludge using high performance liquid chromatography (HPLC); mass spectrometry (MS); thin layer chromatography (TLC); nuclear magnetic resonance (NMR); or gas chromatography (GC).

In the case of investigating the ability to degrade sludge in terms of the turbidity, the presence of ability to degrade sludge indicates a more significantly reduced turbidity after a predetermined time has passed since bacteria for degrading sludge are added to the sludge than a turbidity before the addition. The turbidity after the addition is, for example, 80% or less, preferably 50% or less, and more preferably 30% or less of a turbidity before the addition. In the case of investigating the ability to degrade sludge in terms of the dry weight, the presence of ability to degrade sludge indicates, for example, a more significantly reduced dry sludge weight after the addition than a dry sludge weight of before the addition, and the dry sludge weight after the addition is, for example, 95% or less, and preferably 90% or less of that of before the addition. Generally, bacteria, when having the ability to degrade target microorganisms, have the ability to degrade sludge.

(Bacteria According to the Present Invention)

The bacteria according to the present invention have a 16S rRNA gene comprising the nucleotide sequence represented by SEQ ID NO: 1 with mutations of two bases or less, and have an ability to degrade target microorganisms. In the present description, the 16S rRNA gene is preferably an endogenous 16S rRNA gene that is originally present in bacteria. A 16S rRNA in which a mutation has been artificially induced can be accepted. Examples of the bacteria having a 16S rRNA gene comprising the nucleotide sequence represented by SEQ ID NO: 1 with mutations of two bases or less include bacteria of the genus Brevibacillus, and can be Brevibacillus parabrevis. The bacteria according to the present invention can include the bacteria for degrading sludge according to the present invention.

An embodiment of the bacteria according to the present invention includes bacteria having a 16S rRNA gene comprising the nucleotide sequence represented by SEQ ID NO: 1, and having an ability to degrade target microorganisms.

Examples of the representative strain of bacteria having a 16S rRNA gene comprising the nucleotide sequence represented by SEQ ID NO: 1 include the strain deposited based on Budapest Treaty as Brevibacillus parabrevis (Accession number NITE BP-03020, original deposit date: Sep. 17, 2019) to the Patent Microorganisms Depositary of the National Institute of Technology and Evaluation (NPMD, address: #122, 2-5-8 Kazusakamatari, Kisarazu-shi, Chiba 292-0818). Mycological characteristics of such a strain will be described later.

The bacteria according to the present invention, when compared with the nucleotide sequence represented by SEQ ID NO: 1, can have a 16S rRNA gene comprising a nucleotide sequence having 98.5% or more, 98.8% or more, 99.0% or more, 99.3% or more, 99.5% or more, 99.8% or more, or 99.9% or more identity thereto or homology therewith, or can have a 16S rRNA gene comprising the nucleotide sequence represented by SEQ ID NO: 1.

The bacteria having the nucleotide sequence represented by SEQ ID NO: 1 with mutations of two bases or less can have, when compared with the nucleotide sequence represented by SEQ ID NO: 1, a 16S rRNA gene comprising a nucleotide sequence in which one or two bases are substituted, deleted or added. The above mutation is a mutation by which the expression and functions of the 16S rRNA are not lost.

The bacteria according to the present invention can have a 16S rRNA gene comprising a continuous sequence of about 15 bases or more, preferably about 18 to about 500 bases, more preferably about 18 to about 200 bases, and further preferably about 18 to about 50 bases, that are included in the nucleotide sequence represented by SEQ ID NO: 1, or a nucleotide sequence hybridizable to a complementary sequence thereof under stringent conditions. The stringent conditions refer to conditions under which a non-specific hybrid is not formed and examples include a condition under which one or more washings are carried out at 60° C. with 1×SSC in 0.1% SDS, and preferably at 68° C. with 0.1×SSC in 0.1% SDS.

The ability to degrade target microorganisms of the bacteria according to the present invention varies depending on reaction conditions (kind and concentration of target microorganisms, and composition, temperature, pH, number of bacteria of a solution containing the bacteria). The target microorganism is, for example, a target bacterium, and preferably a target killed bacterium.

For example, the representative strain of the bacteria according to the present invention Bb. parabrevis NITE BP-03020 and killed bacteria of the genus Micrococcus were prepared respectively to have a turbidity (OD660) of 0.2 and mixed in a volume ratio of 1:100 and reacted under a temperature condition of 20 to 35° C. at a pH condition of 6.5 to 8.0 to reduce the turbidity of one week later by about 50%, whereby the bacteria of the genus Micrococcus can be degraded.

The bacteria according to the present invention can be specified by the nucleotide sequence analysis of a 16S rRNA gene and the measurement of ability to degrade target microorganisms of a microorganism of interest (a microorganism to be investigated to identify whether it is the bacterium according to the present invention). Specifically, when a microorganism of interest has a 16S rRNA gene comprising the nucleotide sequence represented by SEQ ID NO: 1 with mutations of two bases or less and has the ability to degrade target microorganisms, such a microorganism can be specified as the bacterium according to the present invention.

The nucleotide sequence analysis of a 16S rRNA gene can be carried out, for example, by the following method. First, using a known method, the genetic DNA is extracted from a microorganism of interest to amplify a 16S rRNA gene. The method for amplifying a 16S rRNA gene is not particularly limited and includes a PCR method that uses universal primers typically used by a person skilled in the art. The amplified product obtained by the PCR method is purified as needed and subjected to a DNA sequencer or the like to determine a nucleotide sequence. The obtained nucleotide sequence is compared with the sequence represented by SEQ ID NO: 1.

Examples of the method for investigating the presence of ability to degrade target microorganisms include a method in which a microorganism of interest and target microorganisms are reacted for a certain time in suitable medium or a buffer solution and then the degradation of the target microorganism in the medium or buffer solution is investigated. The method for investigating the degradation is not particularly limited and examples include a method of measuring a turbidity of a solution containing target microorganisms, a method of detecting target microorganisms using an SLP reagent, a method of detecting the DNA of target microorganisms by PCR, a method of measuring a dry bacterial cell weight of target microorganisms, and a method of detecting degraded products derived from target microorganisms using high performance liquid chromatography (HPLC); mass spectrometry (MS); thin layer chromatography (TLC); nuclear magnetic resonance (NMR); or gas chromatography (GC).

In the case of investigating the degradation of target microorganisms in terms of the turbidity, the presence of ability to degrade target microorganisms indicates a more significantly reduced turbidity after a predetermined time has passed since the bacteria according to the present invention are added to a solution containing the target microorganisms than a turbidity before the addition. The turbidity after the addition is, for example, 80% or less, preferably 50% or less, and more preferably 30% or less of a turbidity before the addition. In the case of investigating the degradation of target microorganisms in terms of the dry bacterial cell weight, the presence of ability to degrade target microorganisms indicates, for example, a more significantly reduced dry bacterial cell weight after the addition than a dry bacterial cell weight of before the addition, and the dry bacterial cell weight after the addition is, for example, 95% or less, and preferably 90% or less of that of before the addition. Generally, bacteria, when having the ability to degrade sludge, have the ability to degrade target microorganisms.

The results of these nucleotide sequence analysis and measurement of ability to degrade target microorganisms enable the conclusion of whether or not a microorganism of interest is the bacterium according to the present invention.

(Microbial Preparation According to the Present Invention)

An embodiment of the microbial preparation according to the present invention comprises the following bacteria (a1).

<Bacteria (a1)>

Bacteria (a1) have a 16S rRNA gene comprising a nucleotide sequence having 90% or more identity when compared with the nucleotide sequence represented by SEQ ID NO: 1.

Bacteria (a1), when compared with the nucleotide sequence represented by SEQ ID NO: 1, can have a 16S rRNA gene comprising a nucleotide sequence having 93% or more, 95% or more, 97% or more, 98% or more, 99% or more, 99.5% or more, or 99.9% or more identity thereto or homology therewith, or can have a 16S rRNA gene comprising the nucleotide sequence represented by SEQ ID NO: 1. Examples of Bacteria (a1) include bacteria of the genus Brevibacillus, and can be Brevibacillus parabrevis. Bacteria (a1) can be the bacteria deposited under Accession number NITE BP-03020.

Further, bacteria (a1) can have a 16S rRNA gene comprising a nucleotide sequence in which one or several bases are substituted, deleted or added when compared with the nucleotide sequence represented by SEQ ID NO: 1. One or several bases can be, for example, 1 to 135 bases, 1 to 100 bases, 1 to 50 bases, 1 to 25 bases, and 1 to 5 bases. The above mutation is a mutation by which the expression and functions of the 16S rRNA are not lost.

Bacteria (a1) can have a 16S rRNA gene comprising a continuous sequence of about 15 bases or more, preferably about 18 to about 500 bases, more preferably about 18 to about 200 bases, and further preferably about 18 to about 50 bases, that are included in the nucleotide sequence represented by SEQ ID NO: 1, or a nucleotide sequence hybridizable to a complementary sequence thereof under stringent conditions.

The nucleotide sequence analysis of a 16S rRNA gene of bacteria (a1) can be carried out in the same manner as for the bacteria according to the present invention described above.

Bacteria (a1) preferably have the ability to degrade target microorganisms. The ability to degrade target microorganisms of bacteria (a1) can be evaluated in the same manner as for the bacteria according to the present invention described above.

Bacteria (a1), due to a good ability to degrade target microorganisms, preferably comprise at least one species of the bacteria selected from Brevibacillus parabrevis, the bacteria for degrading sludge according to the present invention, the bacteria according to the present invention, and the bacteria of the following group A. Bacteria (a1) can be a single bacterium or multiple species of bacteria.

Group A: Brevibacillus invocatus, Brevibacillus centrosporus, Brevibacillus borstelensis, Brevibacillus levickii, Brevibacillus massiliensis, Brevibacillus ginsengisoli, Brevibacillus laterosporus, Brevibacillus fulvus, Brevibacillus fluminis, Brevibacillus sediminis, Brevibacillus thermoruber, Brevibacillus aydinogluensis

The microbial preparation according to the present invention has the ability to degrade sludge. The ability to degrade sludge can be evaluated by the method described in the section for Bacteria for degrading sludge according to the present invention described above. The ability to degrade sludge of the microbial preparation is preferably derived from bacteria (a1) or microorganisms (a2) contained in the microbial preparation, or from a culture thereof. The microbial preparation can degrade sludge at a temperature of 37° C. or less, a temperature of 30° C., a temperature of 25° C., or a temperature of 20° C., and can also degrade sludge in a range from temperatures of 20° C. to 40° C. For example, a turbidity of a solution containing target microorganisms on day 5 since the microbial preparation according to the present invention is added to this solution is 2 times or less as much at a temperature of 25° C. of a turbidity of the solution at a temperature of 30° C., 3 times or less as much at a temperature of 20° C. of a turbidity of the solution at a temperature of 30° C., and preferably 2.5 times or less as much.

The microbial preparation can contain, other than bacteria (a1), a culture of bacteria (a1), microorganisms (a2), additives (b), and a carrier (c). Specific examples of the microbial preparation can include microbial preparations containing bacteria (a1), microorganisms (a2), additives (b), and carrier (c).

<Culture of Bacteria (a1)>

An embodiment of the microbial preparation according to the present invention contains a culture of bacteria (a1). The culture of bacteria (a1) includes, for example, secretion, metabolite of bacteria (a1), proteins, sugars, enzymes produced from bacteria (a1) and liquid medium in which these are suspended. Specifically, the culture includes a culture of bacteria (a1) grown under controlled conditions in predetermined liquid medium or in liquid medium containing a carbon source and a nitrogen source. The microbial preparation for degrading target microorganisms containing the culture of bacteria (a1) can contain viable bacteria of bacteria (a1). The culture of bacteria (a1) can be a supernatant obtained by culturing bacteria (a1). The culture supernatant can be obtained by, for example, removing bacteria (a1) from liquid medium in which bacteria (a1) are cultured by centrifugation, filtration operation or the like. Further, the culture can contain fragments of bacteria (a1). Fragments of bacteria (a1) can also be obtained by subjecting bacteria (a1) to, for example, ultrasonic disintegration, bead grinding, or chemical dissolution treatment. The culture of bacteria (a1) preferably has the ability to degrade a target microorganism.

<Microorganisms (a2)>

Microorganisms (a2) are microorganisms different from bacteria (a1). Microorganisms (a2) are not particularly limited as long as microorganisms do not deprive of the ability to degrade sludge of the microbial preparation. Microorganisms (a2) can be a single bacterium or multiple species of bacteria. Microorganisms (a2) can be bacteria that enhance an ability to proliferate or stability of bacteria (a1), or can be bacteria that enhance the stability of the culture of bacteria (a1). Microorganisms (a2) can be specified by the nucleotide sequence analysis and physiological and biochemical property tests of a 16S rRNA gene of a microorganism. Microorganisms (a2) can have or do no need to have the ability to degrade target microorganisms. The nucleotide sequence analysis of a 16S rRNA gene and the measurement of ability to degrade target microorganisms of a microorganism of interest can be carried out in the same manner as for the bacteria according to the present invention described above.

Microorganisms (a2) can be gram-negative bacteria or gram-positive bacteria, and examples include bacteria of the genus Tumebacillus, bacteria of the genus Paenibacillus, bacteria of the genus bacillus, bacteria of the genus Acetobacter, bacteria of the genus Gluconobacter, bacteria of the genus Lactobacillus, bacteria of the genus Gordonia, bacteria of the genus Microbacterium, bacteria of the genus Rhodococcus, bacteria of the genus Sphingomonas, bacteria of the genus Escherichia, and Saccharomyces cerevisiae.

Microorganisms (a2) preferably contain bacteria having the ability to degrade target microorganisms and, for example, preferably have a 16S rRNA gene comprising a nucleotide sequence having 90% or more identity to the nucleotide sequence represented by SEQ ID NO: 7 and comprise at least one species selected from the group consisting of bacteria having the ability to degrade target microorganisms (hereinafter, also referred to as “bacteria (a21)”), bacteria of the genus bacillus having the ability to degrade target microorganisms, and bacteria of the genus Paenibacillus having the ability to degrade target microorganisms.

Examples of bacteria having a 16S rRNA gene comprising a nucleotide sequence having 90% or more identity to the nucleotide sequence represented by SEQ ID NO: 7 and having the ability to degrade target microorganisms include bacteria of the genus Tumebacillus. Bacteria (a21), when compared with the nucleotide sequence represented by SEQ ID NO: 7, can have a 16S rRNA gene comprising a nucleotide sequence having 93% or more, 95% or more, 98% or more, 99% or more, 99.5% or more, or 99.9% or more identity thereto or homology therewith, or can have a 16S rRNA gene comprising the nucleotide sequence represented by SEQ ID NO: 7. Bacteria (a21) can be the strain deposited based on Budapest Treaty as Tumebacillus sp. (Accession number NITE BP-02779, original deposit date: Sep. 11, 2018) to the Patent Microorganisms Depositary of the National Institute of Technology and Evaluation (NPMD, address: #122, 2-5-8 Kazusakamatari, Kisarazu-shi, Chiba 292-0818). Mycological characteristics of such a strain will be shown in Tables 6 to 9 to be described later.

Bacteria (a21) can be, for example, Tumebacillus algifaecis, Tumebacillus avium, Tumebacillus flagellates, Tumebacillus ginsengisoli, Tumebacillus lipolyticus, Tumebacillus luteolus, Tumebacillus permanentifrigoris, or Tumebacillus soli.

Examples of bacteria of the genus Bacillus having the ability to degrade target microorganisms include Bacillus subtilis, Bacillus amyloliquefaciens, Bacillus sphaericus, and Bacillus licheniformis.

Examples of bacteria of the genus Paenibacillus having the ability to degrade target microorganisms include Paenibacillus polymyxa.

From a viewpoint of the ability to degrade target microorganisms, the percentage of bacteria (a1) based on the total number of bacteria (a1) and microorganisms (a2) can be 0.1% or more, 1% or more, 10% or more, 50% or more, 75% or more, 90% or more, 95% or more, 100% or less, or less than 100%.

The percentage of bacteria (a1) based on the total number of bacteria (a1) and microorganisms (a2) is preferably the percentage of bacteria (a1) in the all bacteria contained in the microbial preparation. Examples of the method for calculating the percentage of bacteria (a1) based on the total number of bacteria (a1) and microorganisms (a2) include methods that calculate by clone library method, next-generation sequencer analysis, and quantitative PCR.

In the method for calculating a percentage of bacteria present by the clone library method and next-generation sequencer analysis, specifically the genomic DNAs are extracted from bacteria (a1) and microorganisms (a2) contained in a microbial preparation to obtain multiple nucleotide sequences of 16S rRNA genes (hereinafter, the number of multiple nucleotide sequences obtained is described as a lead number), and the obtained nucleotide sequences are determined respectively which of bacteria (a1) or microorganisms (a2) they are derived from. Then, the percentage of the number of nucleotide sequences derived from bacteria (a1) based on the lead number is calculated to define the percentage of bacteria (a1) based on the total number of bacteria (a1) and microorganisms (a2).

The next-generation sequencer used for the next-generation sequencer analysis is not particularly limited as long as it can determine a nucleotide sequence by using a DNA fragment as a template and detecting a fluorescence intensity when each base is resynthesized. Examples of the next-generation sequencer include MiSeq, HiSeq 2500 (manufactured by Illumina, Inc.), 5500×I SOLiD™, Ion Proton™, Ion PGM™ (manufactured by Thermo Fisher Scientific), and GS FLX+ (manufactured by Roche Diagnostics).

In the method for calculating a percentage of bacteria present by quantitative PCR, specifically the number of 16S rRNA gene copies of bacteria (a1) and the number of 16S rRNA gene copies of bacteria (a1) and microorganisms (a2) contained in a microbial preparation are respectively calculated. Then, the percentage of the number of 16S rRNA gene copies of bacteria (a1) based on the number of 16S rRNA gene copies of bacteria (a1) and microorganisms (a2) is calculated to define the percentage of bacteria (a1) based on the total number of bacteria (a1) and microorganisms (a2).

The real-time PCR system used for quantitative PCR is not particularly limited as long as it is equipped with a thermal cycler capable of amplifying DNA by PCR and a spectrofluoro-photometer for detecting the amplified products. Examples of the real-time PCR system include StepOnePlus (manufactured by Applied Biosystems), Thermal Cycler Dice Real Time System (manufactured by Takara Bio Inc.), and LightCycler 96 System (manufactured by Roche Diagnostics).

Examples of the master mix used for quantitative PCR include Fast SYBR Green Master Mix, Power SYBR Green Master Mix, SYBR Select Master Mix, and PowerUp SYBR Green Master Mix (manufactured by Thermo Fisher Scientific).

<Additives (b)>

Examples of additives (b) include surfactants, dispersants, adjuvants, and protectants. The kind and concentration of the additives (b) can be suitably determined by conditions under which bacteria (a1) are not killed, or the ability to degrade target microorganisms of such bacteria is not lost.

<Carrier (c)>

Examples of carrier (c) include inorganic fine particle carriers. The inorganic fine particle carrier can be metals and inorganic salts or oxides thereof, can be those containing carbon, or can be those chemically classified as an inorganic matter. The carrier can also be a pure matter with an organic carbon content of less than about 1% or a mixture.

A central particle size of the inorganic fine particle carrier is preferably 1 μm to 100 μm, more preferably 4 μm to 75 μm, and further preferably 13 μm to 25 μm. When a central particle size is within such a range, bacteria (a1) is likely to be supported on the inorganic fine particle carrier. The central particle size herein refers to a median size (D50) in the volume-based particle size distribution by laser diffraction light scattering method. The specific gravity of inorganic fine particle carrier is not particularly limited and preferably 1.2 to 3.5.

The inorganic fine particle carrier can be aggregated using various flocculants as needed for the purpose of improving a yield at the initial stage of culture. Examples of the flocculant include nonionic, cationic, and anionic polymeric flocculants.

Examples of the method for producing the microbial preparation include a method in which bacteria (a1) and an inorganic fine particle carrier are mixed to support bacteria (a1) on the inorganic fine particle carrier and cultured to collect a microbial preparation to be obtained.

For the culture method, any of batch, semi-batch, fed-batch, or continuous mode can be used. For the culture method, for example, a continuous culture mode can be used in which, as described in Japanese Patent Laying-Open No. 9-187272, a concentration of a compound of interest supplied to a vessel in which microorganisms are cultured (hereinafter referred to as a reactor) is logarithmically increased as culture time proceeds from a viewpoint of efficiently preparing microorganisms having slow growth and low bacterial yield.

In the present invention, the formulation technique of microorganisms is not particularly limited as long as the ability to degrade target microorganisms of bacteria (a1) is not lost, and a known formulation technique can be utilized. The form of microbial preparation can be liquid or solid (including an encapsulated form, an agar-like form, a powder form and the like) and can be a frozen form or a freeze-dried form thereof. When the microbial preparation is liquid, a bacterial suspension in which bacteria are suspended in medium, a buffer solution, physiological saline or the like can also be accepted. The liquid can be acidic or neutral. When the microbial preparation is a solid or freeze-dried form, for example, cultured bacteria are concentrated, then suitably dried or freeze-dried to prepare a solid or freeze-dried form. During this procedure, an excipient or the like can be added.

(Sludge Degradation Method)

The degradation method according to the present invention comprises allowing the bacteria for degrading sludge according to the present invention, the bacteria according to the present invention or the microbial preparation according to the present invention to act on sludge. The ability to degrade target microorganisms of the bacteria for degrading sludge according to the present invention, the bacteria according to the present invention and the microbial preparation according to the present invention is useful for treating excess sludge containing target microorganisms, and a volume of excess sludges can be reduced by the degradation method according to the present invention.

The allowing the bacteria for degrading sludge according to the present invention, the bacteria according to the present invention or the microbial preparation according to the present invention to act on sludge refers to, for example, allowing the bacteria for degrading sludge according to the present invention, the bacteria according to the present invention or the microbial preparation according to the present invention, or a solution in which these are suspended, to contact target microorganisms. When the bacteria for degrading sludge according to the present invention, the bacteria according to the present invention or the microbial preparation according to the present invention degrades target microorganisms contained in sludge, the sludge is degraded.

The step of allowing the bacteria for degrading sludge according to the present invention, the bacteria according to the present invention or the microbial preparation according to the present invention to act on sludge is not particularly limited as long as it is conditions under which the bacteria for degrading sludge according to the present invention, the bacteria according to the present invention or bacteria (a1) are not killed, or the ability to degrade target microorganisms of such bacteria is not lost. Such a step can be under the condition of a temperature of 20 to 40° C. or can be under the condition of a temperature of 25 to 30° C. The step can also be under the pH condition of 6.5 to 8.0 or can be under condition of 7.0 to 7.5.

The addition load of the bacteria for degrading sludge according to the present invention, the bacteria according to the present invention or the microbial preparation according to the present invention to sludge can be suitably set in consideration of the kind and concentration of target microorganisms, the volume of reaction system and the like.

In the degradation method according to the present invention, the method for confirming the degradation of sludge is not particularly limited and the confirmation is carried out by a method typically used by a person skilled in the art. Examples of such a confirmation method include the method for evaluating the ability to degrade sludge or the method for evaluating the ability to degrade target microorganisms described above.

(Sludge Degradation Device)

The sludge degradation device is not particularly limited as long as it can reduce excess sludge using the bacteria for degrading sludge according to the present invention, the bacteria according to the present invention or the microbial preparation according to the present invention and examples include sludge treatment device and waste water treatment device from which excess sludge is generated. Further, the bacteria for degrading sludge according to the present invention, the bacteria according to the present invention or the microbial preparation according to the present invention is added to the existing sludge treatment device or waste water treatment device in which excess sludge is present to use as a sludge degradation device.

For degrading excess sludge, chemical treatment such as ozone, alkali and hypochlorous acid, physical treatment such as a bead mill and an ultrasonic crushing, and biological treatment such as using microorganisms that are active under a high temperature condition or under an alkaline condition have been conventionally carried out, however, excess sludge needs to be moved to another tank to carry out these treatments, thereby causing high equipment costs and running costs. The bacteria for degrading sludge according to the present invention, the bacteria according to the present invention and the microbial preparation according to the present invention can be directly put into excess sludge, whereby the cost of investing for new equipment can be saved while the sludge degradation methods that have been conventionally practiced do not need to be changed.

### EXAMPLES

### Experiment 1. Search for Microorganisms Having the Ability to Degrade Target Microorganisms

(Method)

Several strains from a microorganism flora present in the environment (water) were isolated.

(Results)

Each of the isolated strains was investigated for the ability to degrade the bacteria of the genus Micrococcus, and one strain demonstrated the ability to degrade the bacteria of the genus Micrococcus. Hereinafter, the strain that demonstrated the ability to degrade the bacteria of the genus Micrococcus is described as “strain A”.

### Experiment 2. Analysis of Nucleotide Sequence of 16S rRNA Gene of the Strain Having the Ability to Degrade the Bacteria of the Genus Micrococcus

(Materials)
802 Medium: Medium obtained by dissolving 10 g of polypeptone, 2 g of a yeast extract and 1 g of magnesium sulfate heptahydrate in ultrapure water, being adjusted to pH 7.0, then prepared to be 1000 mL and autoclaved
Forward primer for cloning (27f: SEQ ID NO: 2)
Reverse primer for cloning (1492r: SEQ ID NO: 3)
Primers for sequence analysis (339F: SEQ ID NO: 4, 536R: SEQ ID NO: 5, 907F: SEQ ID NO: 6)

(Method)

DNA was extracted from strain A using Easy Extract for DNA (manufactured by AMR Incorporated) and used as a template DNA for amplifying the 16S rRNA gene by PCR.

PCR was carried out under the following conditions. 25.0 μL of 2×PCR buffer for KOD FX (manufactured by TOYOBO CO., LTD.), 10.0 μL of dNTP mix (2 mM), 1.5 μL of forward primer for cloning and reverse primer for cloning (each 10 pmol/μL), 0.58 μL of the template DNA, 10.4 μL of sterilized water, and 1.0 μL of DNA polymerase (KOD FX, 1 U/μL, manufactured by TOYOBO CO., LTD.) were added to a microtube and mixed. The microtube was subjected to a PCR system to carry out the amplification reaction of the template DNA. The reaction was carried out at (1) 94° C. for 2 minutes, (2) 98° C. for 10 seconds, (3) 50° C. for 30 seconds, and (4) 68° C. for 1.5 minutes, and 35 cycles were repeated to carry out steps (2) to (4). The amplified product after PCR was purified.

The purified PCR amplified product in an amount equivalent 150 ng, 0.32 μL of the primers for sequence analysis (10 μM) and 8.0 μL of BigDye Terminator v3.1 (manufactured by Applied Biosystems) were mixed, and sterilized ultrapure water was added thereto to prepare a reaction solution having a fluid volume of 20.0 μL. This reaction solution was subjected to PCR system to carry out the amplification reaction. The reaction was carried out at (1) 96° C. for 1 minutes, (2) 96° C. for 10 seconds, (3) 50° C. for 5 seconds, (4) 60° C. for 4 minutes, and 25 cycles were repeated to carry out steps (2) to (4). The obtained reaction solution was purified, the purified solution was subjected to the DNA sequence analysis (3730×11 DNA Analyzer) to determine the nucleotide sequence of 16S rRNA gene of the template DNA extracted from strain A.

(Results)

The obtained nucleotide sequence was subjected to homology analysis with the International Nucleotide Sequence Databases (DDBJ/ENA(EMBL)/GenBank). This sequence had 99.8% identity with the nucleotide sequence of 16S rRNA gene of Brevibacillus parabrevis IFO12334 among the type strains. However, a microorganism having the 16S rRNA gene completely identical to the obtained nucleotide sequence was not present.

The nucleotide sequence of this 16S rRNA gene is shown in SEQ ID NO: 1. The above suggests that the bacteria having the 16S rRNA gene having the nucleotide sequence represented by SEQ ID NO:1 has the ability to degrade target microorganisms.

### Experiment 3. Morphological Observation and Physiological and Biochemical Property Tests of Bb. Parabrevis Having the 16S rRNA Gene Comprising the Nucleotide Sequence Represented by SEQ ID NO: 1

(Method)

Morphological observation and physiological and biochemical property tests were carried out on strain A. These tests were carried out by morphological observation using an optical microscope, the method of BARROW et al. (Cowan and Steel's Manual for the Identification of Medical Bacteria 3rd Edition 1993, Cambridge University Press.) and API50CHB (manufactured by bioMerieux, Lyon, France). Test results are shown in Table 1 to Table 3.

(Results)

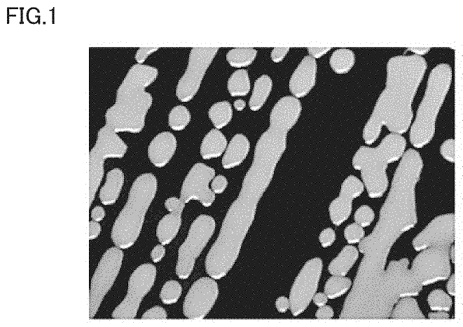

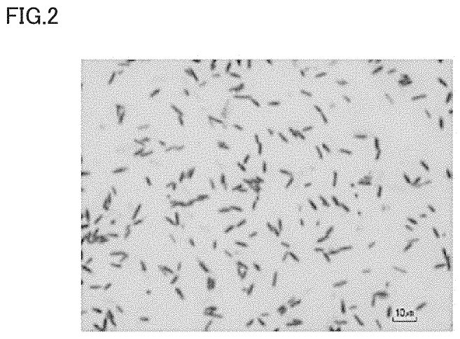

As shown in FIG. 1, a colony of strain A had a circular creamy shape. Further, as shown in FIG. 2, strain A was rod-shaped bacterium and negative in the gram staining. Strain A had different properties in the aspects shown in Table 4 from Bb. parabrevis IFO12334, which has the highest homology with the 16S rRNA gene. For this reason, it suggests that strain A is a strain of new species different from the conventional Bb. parabrevis. Strain A was deposited under Brevibacillus parabrevis NITE BP-03020.

TABLE 1

| Culture temperature (° C.) | | 30 |
|---|---|---|
| Cell morphology | | Rod-shaped |
| | | (1.0 × 2.0 − 3.0 μm) |
| Gram staining | | − |
| Presence or absence of spore | | + Swelling |
| Motility | | + |
| Colony morphology | | Medium: Nutrient Ager |
| | | Culture time: 24 hr |
| | | Diameter: 1 mm |
| | | Color tone: cream |
| | | Shape: circular |
| | | Elevation condition: similar to a |
| | | lens |
| | | Margin: entire margin |
| | | Surface appearance etc.: smooth |
| | | Transparency: opaque |
| | | Viscosity: similar to butter |
| Growth temperature | 37 | + |
| test (° C.) | 45 | + |

| | |
|---|---|
| Catalase reaction | + |
| Oxidase reaction | + |
| Acid/gas production from glucose | −/− |
| (Acid production/gas production) | |
| O/F test (Oxidation/fermentation) | −/− |

+: positive,
−: negative

TABLE 2

| Substrate ingredient | Test results |
|---|---|
| Control | − |
| Glycerol* | − |
| Erythritol* | − |
| D-Arabinose* | − |
| L-Arabinose* | − |
| Ribose* | − |
| D-Xylose* | − |
| L-Xylose* | − |
| Adonitol* | − |
| b-Methyl-D-xylose* | − |
| Galactose* | − |
| Glucose* | − |
| Fructose* | − |
| Mannose* | − |
| Sorbose* | − |
| Rhamnose* | − |
| Dulcitol* | − |
| Inositol* | − |
| Mannitol* | − |
| Sorbitol* | − |
| a-Methyl-D-mannoside* | − |
| a-Methyl-D-glucoside* | − |
| N-Acetylglucosamine* | − |
| Amygdalin* | − |
| Arbutin* | − |
| Aesculin* | + |
| Salicin* | − |
| Cellobiose* | − |
| Maltose* | − |
| Lactose* | − |
| Melibiose* | − |
| Saccharose* | − |
| Trehalose* | − |
| Inulin* | − |
| Melezitose* | − |
| Raffinose* | − |
| Starch* | − |
| Glycogen* | − |
| Xylitol* | − |
| Gentiobiose* | − |
| D-Turanose* | − |
| D-Lyxose* | − |
| D-Tagatose* | − |
| D-Fucose* | − |
| L-Fucose* | − |
| D-Arabitol* | − |
| L-Arabitol* | − |
| Gluconate* | − |
| 2-Ketogluconate* | − |
| 5-Ketogluconate* | − |
| b-Galactosidase** | − |
| Arginine dihydrolase** | − |
| Lysine decarboxylase** | − |
| Ornithine decarboxylase** | − |
| Citric acid utilization** | + |
| H₂S Production** | − |
| Urease** | − |
| Tryptophan deaminase** | − |
| Indole production** | − |
| Acetoin production (VP)** | − |
| Gelatinase** | + |
| Nitrate reduction** | − |

*Fermentability test,
**Biochemical test
+: positive,
−: negative

TABLE 3

| Test items | Test results |
|---|---|
| Growth | |
| 50° C. | + |
| 55° C. | − |
| 20° C. | + |
| Anaerobic | − |
| Hydrolysis | |
| Casein | + |
| Tween80 (lipase activity) | + |
| Starch | − |
| Assimilation | |
| Glucose | + |
| Trehalose | + |
| Glycerol | + |
| Mannitol | + |
| Maltose | + |

TABLE 4

| | Brevibacillus parabrevis | |
|---|---|---|
| Substrate ingredient | IFO12334 | Strain A |
| Glucose | Oxidized | Not oxidized |
| Mannitol | Oxidized | Not oxidized |
| Aesculin | Negative | Positive |
| Citric acid utilization | Negative | Positive |
| Gelatinase | Negative | Positive |
| Nitrate reduction | Reduced | Not reduced |

### Experiment 4. Degradation Ability Evaluation of Bb. Parabrevis NITE BP-03020 on a Target Microorganism

(Materials)
802 Medium: Medium obtained by dissolving 10 g of polypeptone, 2 g of a yeast extract and 1 g of magnesium sulfate heptahydrate in ultrapure water, being adjusted to pH 7.0, then prepared to be 1000 mL and autoclaved
Solution A: Solution obtained by dissolving 4.35 g of dipotassium hydrogenphosphate, 1.70 g of monopotassium dihydrogen phosphate, 8.92 g of di-sodium hydrogenphosphate 12-Water and 0.34 g of ammonium chloride in ultrapure water, then being prepared to be 200 mL and autoclaved
Solution B: Solution obtained by dissolving 4.50 g of magnesium sulfate heptahydrate in ultrapure water, then being prepared to be 200 mL and autoclaved
Solution C: Solution obtained by dissolving 5.50 g of calcium chloride anhydrous in ultrapure water, then being prepared to be 200 mL and autoclaved
Solution D: Solution obtained by dissolving 0.05 g of iron chloride hexahydrate in ultrapure water, then being prepared to be 200 mL and filter sterilized with a 0.2 μm syringe filter

(Method)

Micrococcus (target microorganism) was cultured in 802 medium at a temperature of 25° C., collected and washed, and mixed in such a way as to have a turbidity (OD660) of 0.2 to 986 mL of ultrapure water and autoclaved to obtain a substrate solution. 3.0 mL of a solution A, 3.0 mL of a solution B, 3.0 mL of a solution C, 3.0 mL of a solution D and 1.8 mL of 1% phosphoric acid were mixed in 986 mL of the substrate solution to obtain a target microorganism-containing solution having inorganic medium as a solvent. The target microorganism contained in the target microorganism-containing solution is target killed bacterium.

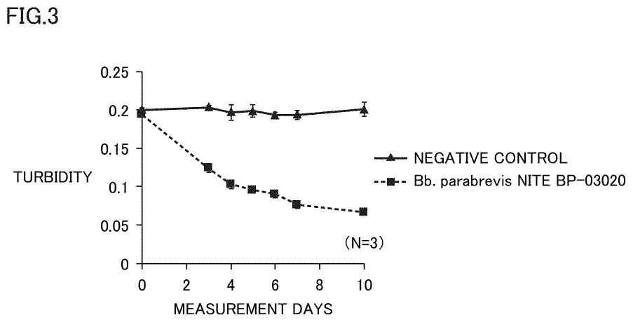

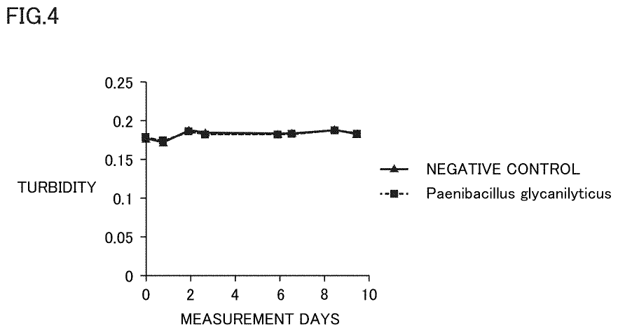

Bb. parabrevis NITE BP-03020 was seeded in 802 medium and cultured at 30° C. for 24 to 48 hours. After the culture, 50 μL of the Bb. parabrevis NITE BP-03020 culture solution was added to 5.0 mL of the target microorganism-containing solution, shaken at 25° C. and 200 rpm and measured over time for the turbidity (OD660) of the test tube with an easy operation turbidity meter (easy operation OD monitor miniphoto 518R, manufacture by TAITEC Corporation). The results are shown in FIG. 3. Further, 50 μL of a culture solution of Paenibacillus glycanilyticus cultured by the same method was added to 50 mL of the target microorganism-containing solution to measure a turbidity, and the results thereof are shown in FIG. 4. Note that for the negative control, the turbidity (OD660) of a target microorganism-containing solution to which the Bb. parabrevis NITE BP-03020 culture solution was not added was used.

As shown in FIG. 3, the negative control had substantially no change in the turbidity of the target microorganism-containing solution, whereas the target microorganism-containing solution to which the Bb. parabrevis NITE BP-03020 culture solution was added had decreases in the turbidity by 50% or less before the addition. Further, as shown in FIG. 4, the target microorganism-containing solution to which the Paenibacillus sp. culture solution was added had no decrease in the turbidity. The above results reveled that Bb. parabrevis has the ability to degrade target microorganisms.

### Experiment 5. Degradation Ability Evaluation of Microbial Preparations Containing the Bb. Parabrevis NITE BP-03020 Cultures on a Target Microorganism

### (Method)

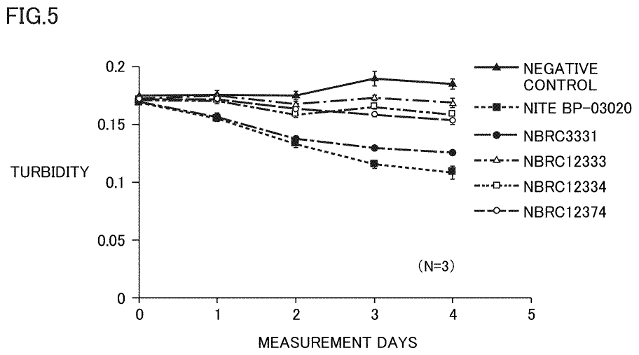

Bb. parabrevis NITE BP-03020, Bb. parabrevis NBRC3331, Bb. parabrevis NBRC12333, Bb. parabrevis NBRC12334, and Bb. parabrevis NBRC12374 were respectively seeded in 802 medium and cultured at 30° C. for 24 to 48 hours. After the culture, the culture solution was filtered with a 0.2 μm syringe filter to obtain a culture supernatant (a culture). 50 μL of the culture supernatant was added to 5.0 mL of the target microorganism-containing solution, shaken at 25° C. and 200 rpm and measured over time for the turbidity (OD660) of the test tube with an easy operation turbidity meter (easy operation OD monitor miniphoto 518R, manufacture by TAITEC Corporation). The results are shown in FIG. 5. Note that for the negative control, the turbidity (OD660) of a target microorganism-containing solution to which the Bb. parabrevis culture supernatant was not added was used. The same solution as Experiment 4 was used for the target microorganism-containing solution. Bb. parabrevis NBRC3331, Bb. parabrevis NBRC12333, Bb. parabrevis NBRC12334, and Bb. parabrevis NBRC12374 were obtained from the National Institute of Technology and Evaluation.

(Results)

Each of the Bb. parabrevis strains other than Bb. parabrevis NITE BP-03020 also had decreases in the turbidity of the target microorganism-containing solution when the culture supernatant was added. Thus, it was revealed that Bb. parabrevis other than Bb. parabrevis NITE BP-03020 also had the ability to degrade a target microorganism. Further, it was revealed that the cultures of Bb. parabrevis also had the ability to degrade a target microorganism.

Further, the target microorganism-containing solution to which the Bb. parabrevis NITE BP-03020 culture supernatant was added had a more decrease in the turbidity than the target microorganism-containing solutions to which other Bb. parabrevis strain culture supernatants were added. It was revealed that Bb. parabrevis NITE BP-03020 was superior in the ability to degrade target microorganism to other Bb. parabrevis strains.

### Experiment 6. Degradation Ability Evaluation of the Microbial Preparation Containing the Bb. Parabrevis NITE BP-03020 Culture on Multiple Target Microorganisms

(Materials)
LB Liquid medium: Medium obtained by dissolving LB Broth, 1.1 G PER TABLET (manufactured by SIGMA) in a proportion of 10 tablets in 500 mL of ultrapure water and being autoclaved
802 Medium: Same as Experiment 4
Medium for bacteria: Medium obtained by dissolving 20 g of glucose, 5 g of polypeptone, 3 g of a yeast extract, 3 g of a meat extract, 2 g of ammonium sulfate, 1 g of monopotassium dihydrogen phosphate and 0.5 g of magnesium sulfate heptahydrate in ultrapure water, being adjusted to pH 7.0, then prepared to be 1000 mL and autoclaved
YM Medium: Medium obtained by dissolving 20 g of glucose, 5 g of peptone, 3 g of a yeast extract, and 3 g of a malt extract in ultrapure water, and autocraved

The target microorganisms shown in Table 5 were cultured in the media and at culture temperatures shown in the same table, collected and washed, and mixed in such a way as to have a turbidity (OD660) of 0.2 to 0.3 to 986 mL of ultrapure water and autoclaved to obtain a substrate solution. The same Solution A to Solution D and phosphoric acid solution as in Experiment 4 were mixed in the substrate solution to obtain the target microorganism-containing solutions for various species of bacteria (killed bacteria) as the target microorganisms.

TABLE 5

| | | Culture temperature |
|---|---|---|
| Target microorganisms | Medium | (° C.) |
| Micrococcus lysodeikticus | 802 Medium | 25 |
| Saccharomyces cerevisiae | YM Medium | 37 |
| Escherichia coli | LB Medium | 37 |
| Brevibacillus parabrevis | 802 Medium | 30 |
| Gluconobacter oxydans | Medium for | 30 |
| | bacteria | |

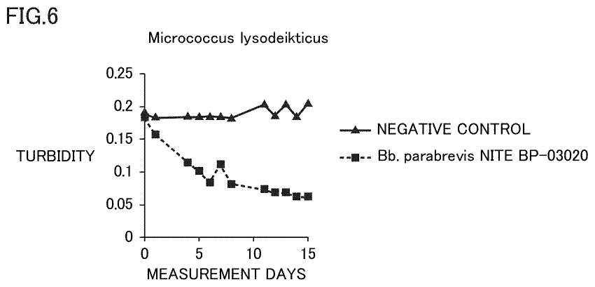

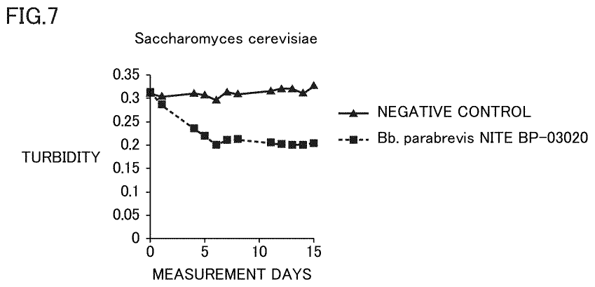

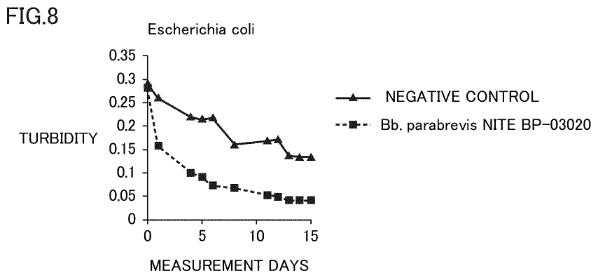

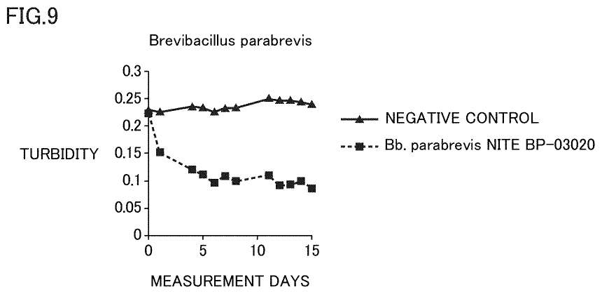

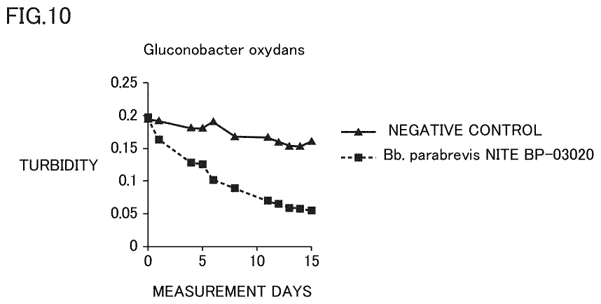

The Bb. parabrevis NITE BP-03020 culture supernatant was added to each of the target microorganism-containing solutions by the same method as in Experiment 5 in the exception of the kind of target microorganism-containing solutions to measure over time for the turbidity. The results are shown in FIG. 6 to FIG. 10.

(Results)

All target microorganism-containing solutions to which the Bb. parabrevis NITE BP-03020 culture supernatant was added had more decreases in the turbidity than the negative control, revealing that Bb. parabrevis and the culture thereof were capable of degrading various species of target microorganisms.

### Experiment 7. Sludge Degradation Ability Evaluation of the Microbial Preparation Containing the Bb. Parabrevis NITE BP-03020 Culture

(Materials)

Excess sludge, after washed, was mixed in such a way as to have a turbidity (OD660) of 0.2 to 986 mL of ultrapure water and being autoclaved to obtain an excess sludge substrate solution. The target microorganism in the excess sludge substrate solution is a killed bacterium. An excess sludge-containing solution, in which 3.0 mL of Solution A, 3.0 mL of Solution B, 3.0 mL of Solution C, 3.0 mL of Solution D, and 1.8 mL of 1% phosphoric acid were mixed with 986 mL of the excess sludge substrate solution, was prepared.

(Method)

The Bb. parabrevis NITE BP-03020 culture supernatant was added to the excess sludge-containing solution by the same method as in Experiment 5 in the exception that the excess sludge-containing solution was used as the solution containing the target microorganism to measure over time for the turbidity of the excess sludge-containing solution.

(Results)

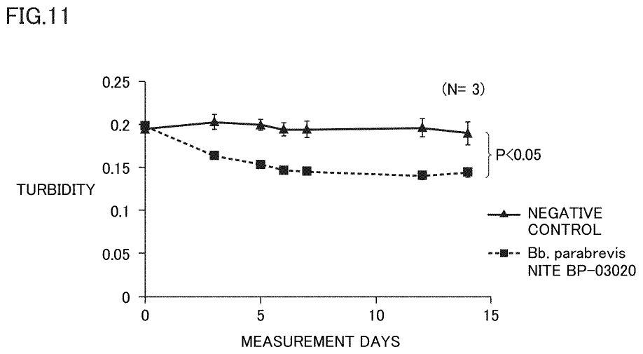

As show in FIG. 11, the excess sludge-containing solution had a decrease in the turbidity when the Bb. parabrevis NITE BP-03020 culture supernatant was added. Thus, it was revealed that the Bb. parabrevis NITE BP-03020 culture had the ability to degrade sludge.

### Experiment 8. Temperature Sensitivity Evaluation on the Ability to Degrade a Target Microorganism of the Microbial Preparation Containing the Bb. Parabrevis NITE BP-03020 Culture

(Method)

A turbidity of the target microorganism-containing solution was measured over time by the same method as in Experiment 5 in the exception that the shaking temperature after the Bb. parabrevis NITE BP-03020 culture supernatant was added to the target microorganism-containing solution was changed. The shaking temperatures were 20° C., 25° C., 30° C. or 37° C.

(Results)

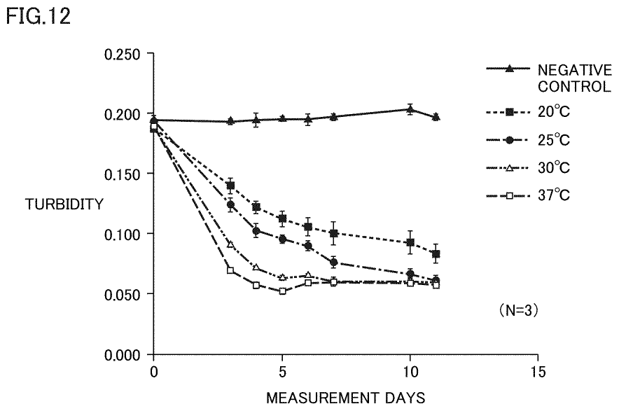

As shown in FIG. 12, the target microorganism-containing solution to which the Bb. parabrevis NITE BP-03020 culture supernatant was added had decreases in the turbidity at the shaking temperatures of 20° C. to 37° C. It was revealed that the microbial preparation containing the Bb. parabrevis NITE BP-03020 culture had the ability to degrade a target microorganism even at a temperature of 20° C.

### Experiment 9. Sludge Degradation Ability Evaluation of the Complex Microbial Preparation Containing Each Culture Supernatant of Bb. Parabrevis NITE BP-03020, B. subtilis Subsp. inaquosorum DSM022148, and Tumebacillus Sp. NITE BP-02779

### (Material)

Excess sludge, after washed, was mixed in such a way as to have a turbidity (OD660) of 0.15 to 986 mL of ultrapure water and autoclaved to obtain an excess sludge substrate solution. The target microorganism in the excess sludge substrate solution is a killed bacterium. An excess sludge-containing solution, in which 3.0 mL of Solution A, 3.0 mL of Solution B, 3.0 mL of Solution C, 3.0 mL of Solution D, and 1.8 mL of 1% phosphoric acid were mixed in 986 mL of the excess sludge substrate solution, was prepared.

B. subtilis subsp. inaquosorum DSM022148 used was a bacterium isolated from the environment. The nucleotide sequence of the 16S rRNA gene of B. subtilis subsp. inaquosorum DSM022148 is shown in SEQ ID NO: 8. The identity of the nucleotide sequence of 16S rRNA gene of Bb. parabrevis NITE BP-03020 to Tumebacillus sp. NITE BP-02779 was 87%, and the identity of the nucleotide sequence of 16S rRNA gene of Bb. parabrevis NITE BP-03020 to B. subtilis subsp. inaquosorum DSM022148 was 89%.

(Method)

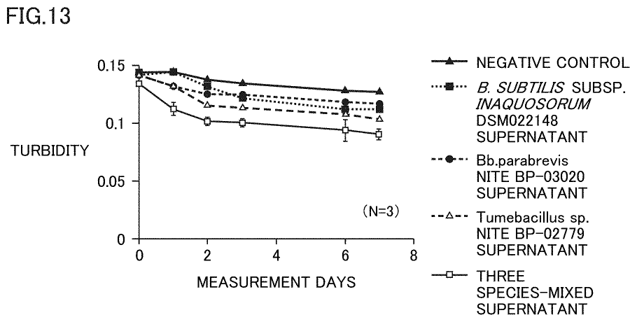

Bb. parabrevis NITE BP-03020 was seeded in 802 medium and cultured at 30° C. for 24 to 48 hours. B. subtilis subsp. inaquosorum DSM022148 was seeded in 802 medium and cultured at 37° C. for 24 to 48 hours. Tumebacillus sp. NITE BP-02779 was seeded in R2A medium and cultured at 25° C. for 24 to 48 hours. After the completion of culture, the culture solutions were filtered with a 0.2 μm syringe filter to obtain culture supernatants (cultures). 50 μL of each culture supernatant was added to 5.0 mL of the excess sludge-containing solution, shaken at 25° C. and 200 rpm, and measured over time for the turbidity (OD660) of the test tube with an easy operation turbidity meter (easy operation OD monitor miniphoto 518R, manufacture by TAITEC Corporation). In the three species-mixed supernatant-added solution, 16.7 μL each of the culture supernatants was added. The results are shown in FIG. 13. Note that for the negative control, the turbidity (OD660) of a target microorganism-containing solution to which the culture supernatants were not added was used.

(Results)

As shown in FIG. 13, more significant decreases in the turbidity of the excess sludge-containing solution were observed when each of the microorganisms was mixed and added than the culture supernatant of Bb. parabrevis NITE BP-03020, B. subtilis subsp. inaquosorum DSM022148, and Tumebacillus sp. NITE BP-02779 was singly added. Thus, it was revealed that the complex microbial preparation containing the Bb. parabrevis NITE BP-03020 culture and two other microorganisms also had the ability to degrade sludge.

### Reference Experiment 1. Search for Microorganisms Having the Ability to Degrade Target Microorganisms

(Method)

Using medium containing bacteria of the genus Micrococcus as a carbon source, a microorganism flora present in the environment (water) was cultured to subject the microorganisms that degrade the bacteria of the genus Micrococcus to enrichment culture. Then, several strains that grew well were isolated from the enriched microorganism flora.

(Results)

Each of the isolated strains was investigated for the ability to degrade the bacteria of the genus Micrococcus, and one strain demonstrated the ability to degrade the bacteria of the genus Micrococcus. Hereinafter, the strain that demonstrated the ability to degrade the bacteria of the genus Micrococcus may be described as “strain B”.

### Reference Experiment 2. Analysis of Nucleotide Sequence of 16S rRNA Gene of the Strain Having the Ability to Degrade the Bacteria of the Genus Micrococcus

(Materials)
R2A Medium: Medium obtained by dissolving R2A Broth, DAIGO (manufactured by Nihon Pharmaceutical Co., Ltd.) in a proportion of 3.2 g in 1000 mL of ultrapure water and being autoclaved
Forward primer for cloning (27f: SEQ ID NO: 2)
Reverse primer for cloning (1492r: SEQ ID NO: 3)
Primers for sequence analysis (339F: SEQ ID NO: 4, 536R: SEQ ID NO: 5, 907F: SEQ ID NO: 6)

(Method)

DNA was extracted from strain B using Easy Extract for DNA (manufactured by AMR Incorporated) and used as a template DNA for amplifying the 16S rRNA gene by PCR.

PCR was carried out under the following conditions. 25.0 μL of 2×PCR buffer for KOD FX (manufactured by TOYOBO CO., LTD.), 10.0 μL of dNTP mix (2 mM), 1.5 μL of forward primer for cloning and reverse primer for cloning (each 10 pmol/μL), 0.58 μL of the template DNA, 10.4 μL of sterilized water, and 1.0 μL of DNA polymerase (KOD FX, 1 U/μL, manufactured by TOYOBO CO., LTD.) were added to a microtube and mixed. The microtube was subjected to a PCR system to carry out the amplification reaction of the template DNA. The reaction was carried out at (1) 94° C. for 2 minutes, (2) 98° C. for 10 seconds, (3) 50° C. for 30 seconds, and (4) 68° C. for 1.5 minutes, and 35 cycles were repeated to carry out steps (2) to (4). The amplified product after PCR was purified.

The purified PCR amplified product in an amount equivalent 150 ng, 0.32 μL of the primers for sequence analysis (10 μM) and 8.0 μL of BigDye Terminator v3.1 (manufactured by Applied Biosystems) were mixed, and sterilized ultrapure water was added thereto to prepare a reaction solution having a fluid volume of 20.0 μL. This reaction solution was subjected to PCR system to carry out the amplification reaction. The reaction was carried out at (1) 96° C. for 1 minutes, (2) 96° C. for 10 seconds, (3) 50° C. for 5 seconds, (4) 60° C. for 4 minutes, and 25 cycles were repeated to carry out steps (2) to (4). The obtained reaction solution was purified, the purified solution was subjected to the DNA sequence analysis (3730×1 DNA Analyzer) to determine the nucleotide sequence of 16S rRNA gene of the template DNA extracted from strain B.

(Results)

The obtained nucleotide sequence was subjected to homology analysis with the International Nucleotide Sequence Databases (DDBJ/ENA(EMBL)/GenBank). This sequence had 98.1% identity to the nucleotide sequence of 16S rRNA gene of Tumebacillus permanentifrigoris Eurl_9.5 among the type strains. However, a microorganism having the 16S rRNA gene completely identical to the obtained nucleotide sequence was not present.

The obtained nucleotide sequence of the 16S rRNA gene is shown in SEQ ID NO: 7. This suggests that the bacteria having the 16S rRNA gene having the nucleotide sequence represented by SEQ ID NO: 7 has the ability to degrade microorganisms.

### Reference Experiment 3. Morphological Observation and Physiological and Biochemical Property Tests of the Bacteria of the Genus Tumebacillus Having the 16S rRNA Gene Having the Nucleotide Sequence Represented by SEQ ID NO: 7

(Method)

Morphological observation and physiological and biochemical property tests were carried out on strain B. These tests were carried out by morphological observation using an optical microscope, the method of BARROW et al. (Cowan and Steel's Manual for the Identification of Medical Bacteria 3rd Edition 1993, Cambridge University Press.) and API50CHB (manufactured by bioMerieux, Lyon, France). Tests results are shown in Table 6 to Table 9.

(Results)

Strain B did not glow at 10° C. but this property was not found in Tumebacillus permanentifrigoris Eurl_9.5, which has the highest homology with the 16S rRNA gene. For this reason, it is suggested that strain B is a new species different from the conventional Tumebacillus. Strain B was deposited under Tumebacillus sp. NITE BP-02779.

TABLE 6

| Test items | | Results |
|---|---|---|
| Culture temperature (° C.) | | 25 |
| Cell morphology | | Rod-shaped (0.8 − 0.9 × 3.0 − 5.0 μm) |
| Gram staining | | + |
| Presence or absence of spore | | + |
| Motility | | − |
| Colony morphology | | Medium: R2A Ager |
| | | Culture time: 48 hr |
| | | Diameter: 1-2 mm |
| | | Color tone: yellow |
| | | Shape: circular |
| | | Elevation condition: similar to a lens |
| | | (elevated in the center) |
| | | Margin: undulate |
| | | Surface appearance etc.: smooth |
| | | Transparency: opaque |
| | | Viscosity: similar to butter |
| Growth temperature | 37 | + |
| test (° C.) | 45 | − |

| | |
|---|---|
| Catalase reaction | +w |
| Oxidase reaction | − |
| Acid/gas production from | −/− |
| glucose (Acid production/ | |
| gas production) | |
| O/F test (Oxidation/ | −/− |
| fermentation) | |

+: positive,
−: negative,
+w: weak reaction

TABLE 7

| | Test |
|---|---|
| | results |
| Test items | |
| (Fermentability test) | |
| Control | − |
| Glycerol | − |
| Erythritol | − |
| D-Arabinose | − |
| L-Arabinose | − |
| Ribose | − |
| D-Xylose | − |
| L-Xylose | − |
| Adonitol | − |
| β-Methyl-D-Xylose | − |
| Galactose | − |
| Glucose | − |
| Fructose | − |
| Mannose | − |
| Sorbose | − |
| Rhamnose | − |
| Dulcitol | − |
| Inositol | − |
| Mannitol | − |
| Sorbitol | − |
| α-Methyl-D- | − |
| mannoside | |
| α-Methyl-D- | − |
| glucoside | |
| N-Acetylglucosamine | − |
| Amygdalin | − |
| Arbutin | − |
| Aesculin | − |
| Salicin | − |
| Cellobiose | − |
| Maltose | − |
| Lactose | − |
| Melibiose | − |
| Saccharose | − |
| Trehalose | − |
| Inulin | − |
| Melezitose | − |
| Raffinose | − |
| Starch | − |
| Glycogen | − |
| Xylitol | − |
| Gentiobiose | − |
| D-Turanose | − |
| D-Lyxose | − |
| Substrate ingredient | |
| (Fermentability test) | |
| D-Tagatose | − |
| D-Fucose | − |
| L-Fucose | − |
| D-Arabitol | − |
| L-Arabitol | − |
| Gluconate | − |
| 2-Ketogluconate | − |
| 5-Ketogluconate | − |

+: positive,
−: negative

TABLE 8

| Substrate ingredient | |
|---|---|
| (Biochemical test) | Test results |
| β-Galactosidase | − |
| Arginine dihydrolase | − |
| Lysine decarboxylase | − |
| Ornithine decarboxylase | − |
| Citric acid utilization | − |
| H₂S Production | − |
| Urease | − |
| Tryptophan deaminase | − |
| Indole production | − |
| Acetoin production (VP) | − |
| Gelatinase | − |
| Nitrate reduction | − |

+: positive,
−: negative

TABLE 9

| Test items | Test results |
|---|---|
| Growth at 10° C. | − |
| Growth at pH 9.0 | + |
| Hydrolysis of starch | + |

+: positive,
−: negative

### Experiment 4. Degradation Ability Evaluation of Tumebacillus Sp. NITE BP-02779 on Target Bacteria (Killed Bacteria)

(Materials)
R2A Medium: Medium obtained by dissolving R2A Broth, DAIGO (manufactured by Nihon Pharmaceutical Co., Ltd.) in a proportion of 3.2 g in 1000 mL of ultrapure water and being autoclaved
LB Liquid medium: Medium obtained by dissolving LB Broth, 1.1 G PER TABLET (manufactured by SIGMA) in a proportion of 10 tablets in 500 mL of ultrapure water and being autoclaved
802 Medium: Medium obtained by dissolving 10 g of polypeptone, 2 g a yeast extract and 1 g of magnesium sulfate heptahydrate in ultrapure water, being adjusted to pH 7.0, then prepared to be 1000 mL and autoclaved
Medium for bacteria: Medium obtained by dissolving 20 g of glucose, 5 g of polypeptone, 3 g of a yeast extract, 3 g of a meat extract, 2 g of ammonium sulfate, 1 g of monopotassium dihydrogen phosphate and 0.5 g of magnesium sulfate heptahydrate in ultrapure water, being adjusted to pH 7.0, then prepared to be 1000 mL and autoclaved
Target bacterium (killed bacterium)-containing inorganic medium: Medium obtained by mixing 986 mL of a substrate solution, 3.0 mL of a solution A, 3.0 mL of a solution B, 3.0 mL of a solution C, 3.0 mL of a solution D and 1.8 mL of 1% phosphoric acid

Note that the above substrate solution and solutions A to D used were as follows.
Substrate solution: Solution obtained by culturing a target bacterium shown in Table 10 under the conditions shown in the same table, being collected and washed, and mixed in such a way as to have a turbidity (OD660) of 0.2 to 986 mL of ultrapure water and autoclaved
Solution A: Solution obtained by dissolving 4.35 g of dipotassium hydrogenphosphate, 1.70 g of monopotassium dihydrogen phosphate, 8.92 g of di-sodium hydrogenphosphate 12-Water and 0.34 g of ammonium chloride in ultrapure water, then being prepared to be 200 mL and autoclaved
Solution B: Solution obtained by dissolving 4.50 g of magnesium sulfate heptahydrate in ultrapure water, then being prepared to be 200 mL and autoclaved
Solution C: Solution obtained by dissolving 5.50 g of calcium chloride anhydrous in ultrapure water, then being prepared to be 200 mL and autoclaved
Solution D: Solution obtained by dissolving 0.05 g of iron chloride hexahydrate in ultrapure water, then being prepared to be 200 mL and filter sterilized with a 0.2 μm syringe filter

TABLE 10

| | | Culture temperature |
|---|---|---|
| Target bacteria | Medium | (° C.) |
| Micrococcus | 802 Medium | 25 |
| Bacillus | Medium for bacteria | 25 |
| Staphylococcus | R2A Medium | 30 |
| Lactobacillus | 802 Medium | 25 |
| Paenibacillus | Medium for bacteria | 25 |
| Escherichia | LB Medium | 37 |
| Acetobacter | 802 Medium | 25 |

(Method)

Tumebacillus sp. NITE BP-02779 was seeded in R2A medium and cultured at 25° C. for 24 hours to 48 hours.

After the completion of culture, 50 μL of the Tumebacillus sp. NITE BP-02779 culture solution and 5.0 mL of the target bacterium (killed bacterium)-containing inorganic medium were added to a test tube, reacted at 25° C. and 200 rpm and measured over time for the turbidity (OD660) of the test tube with an easy operation turbidity meter (easy operation OD monitor miniphoto 518R, manufacture by TAITEC Corporation). The number of days that has elapsed on which the turbidity (OD660) of the target bacterium (killed bacterium)-containing inorganic medium showed 50% of the turbidity (OD660) of negative control was calculated and the presence or absence of degradation ability was decided by the following criteria.

A: 0 Days or more and less than 2.0 days

B: 2.0 Days or more and less than 5.0 days

C: 5.0 Days or more

Note that for the negative control, the turbidity (OD660) of target bacterium (killed bacterium)-containing inorganic medium to which Tumebacillus sp. NITE BP-02779 was not added was used.

(Results)

The results are shown in Table 11. When Tumebacillus sp. NITE BP-02779 was added, decreases in the turbidity of target bacterium (killed bacterium)-containing inorganic medium were observed. Thus, Tumebacillus sp. NITE BP-02779 was capable of degrading the target microorganisms (target killed bacteria).

TABLE 11

| Target bacteria | | | |
|---|---|---|---|
| (killed bacteria) | Results | Cell morphology | Gram staining |
| Micrococcus | A (0.7 days) | Coccus | Positive |
| Bacillus | A (0.8 days) | Rod-shaped | Positive |
| Staphylococcus | A (1.9 days) | Coccus | Positive |
| Lactobacillus | A (1.9 days) | Rod-shaped | Positive |
| Paenibacillus | A (1.1 days) | Rod-shaped | Positive |
| Escherichia | A (0.6 days) | Rod-shaped | Negative |
| Acetobacter | A (0.7 days) | Rod-shaped | Negative |

### Reference Experiment 5. Degradation Ability Evaluation of Tumebacillus Sp. NITE BP-02779 on Target Bacteria (Viable Bacteria)

(Materials)
Target bacterium (viable bacterium)-containing inorganic medium: Solution obtained by mixing 986 mL of sterilized water, 3.0 mL of solution A, 3.0 mL of solution B, 3.0 mL of solution C, 3.0 mL of solution D, 1.8 mL of 1% phosphoric acid and target bacterium (viable bacterium)

Note that the same solutions as in Reference Experiment 4 were used as solutions A to D. Further, the target bacterium (viable bacterium) was mixed so that the turbidity (OD660) of the target bacterium (viable bacterium)-containing inorganic medium was 0.2.

(Method)

The degradation ability of Tumebacillus sp. NITE BP-02779 on the target bacteria (viable bacteria) was evaluated by the same method as in Reference Experiment 4 in the exception that the target bacterium (viable bacterium)-containing inorganic medium was used.

(Results)

The results are shown in Table 12. When Tumebacillus sp. NITE BP-02779 was added, decreases in the turbidity of the target bacterium (viable bacterium)-containing inorganic medium were observed. Thus, Tumebacillus sp. NITE BP-02779 was capable of degrading the target bacteria (viable bacteria).

TABLE 12

| Target bacteria | | | |
|---|---|---|---|
| (viable bacteria) | Results | Cell morphology | Gram staining |
| Micrococcus | B (2.0 days) | Coccus | Positive |
| Staphylococcus | A (1.5 days) | Coccus | Positive |

### Reference Experiment 6. Degradation Ability Evaluation of Tumebacillus Sp. NITE BP-02779 on Excess Sludge (Killed Bacteria) (1)

Excess sludge (killed bacteria)-containing inorganic medium: Medium obtained by mixing 986 mL of a substrate solution, 3.0 mL of solution A, 3.0 mL of solution B, 3.0 mL of solution C, 3.0 mL of solution D and 1.8 mL of 1% phosphoric acid

Substrate solution: Solution obtained by washing the excess sludge, then mixing the sludge in such a way as to have a turbidity (OD660) of 0.2 to 986 mL of ultrapure water and being autoclaved

The same solutions as in Reference Experiment 4 were used in the exception that the above substrate solution was used.

(Method)

The degradation ability of Tumebacillus sp. NITE BP-02779 on the excess sludge (killed bacteria) was evaluated by the same method as in Reference Experiment 4 in the exception that the excess sludge (killed bacteria)-containing inorganic medium was used.

(Results)

The results are shown in Table 13. When Tumebacillus sp. NITE BP-02779 was added, a decrease in the turbidity of excess sludge (killed bacteria) was observed. Thus, Tumebacillus sp. NITE BP-02779 was capable of degrading the excess sludge (killed bacteria).

TABLE 13

| Target bacteria | | | |
|---|---|---|---|
| (killed bacteria) | Results | Cell morphology | Gram staining |
| Excess sludge | B (3.2 days) | − | − |

### Reference Experiment 7. Degradation Ability Evaluation of Tumebacillus Sp. NITE BP-02779 on Excess Sludge (Killed Bacteria) (2)

(Materials)

The same materials as in Reference Experiment 6 were used.

(Method)

The reaction was carried out by the same method as in Reference Experiment 4. The quintuplicate reaction was serially carried out, and the total amount of excess sludge remained in a test tube was collected on day 4 since the reaction had started. The collected excess sludge was dried and then measured for a dry weight of the excess sludge to carry out the significance test (t-test, one-sided) between the negative control group and the Tumebacillus sp. NITE BP-02779 addition group.

(Result)

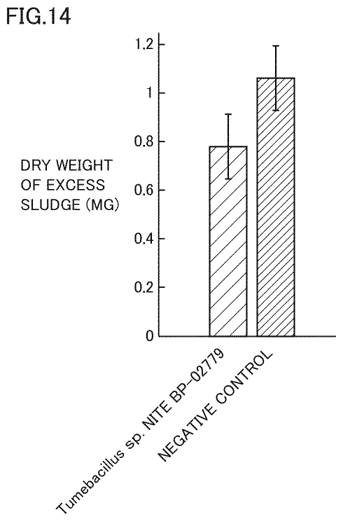

The result is shown in FIG. 14. A significant decrease (p<0.01) in the dry weight of excess sludge was observed in the Tumebacillus sp. NITE BP-02779 addition group in comparison with the negative control group. Thus, the excess sludge was reducible by the addition of Tumebacillus sp. NITE BP-02779.

### Reference Experiment 8. Degradation Ability Evaluation of Various Bacteria on the Bacteria of the Genus Micrococcus

(Materials)

For target bacterium-containing inorganic medium, target bacterium (killed bacterium)-containing inorganic medium that contains the bacteria of the genus Micrococcus used in Reference Experiment 4 as the target bacteria was prepared. The bacteria shown in Table 14 were prepared as the bacteria to be evaluated for the presence or absence of microorganism degradation ability.

TABLE 14

Tumebacillus sp. NITE BP-02779
Tumebacillus algifaecis NBRC108765t
Bacillus alvei IFO 3343t
Bacillus badius ATCC 14574t
Bacillus brevis IFO 3331
Bacillus brevis JCM 2503t
Bacillus cereus JCM 2152t
Bacillus cereus var. juroi ATCC 21281
Bacillus circulans ATCC 13403
Bacillus circulans IFO 3329
Bacillus coagulans JCM 2257t
Bacillus firmus JCM 2512t
Bacillus lentus JCM 2511t
Bacillus licheniformis ATCC 14594
Bacillus licheniformis ATCC 27811
Bacillus licheniformis IFO 12195
Bacillus licheniformis IFO 12197
Bacillus licheniformis IFO 12200t
Bacillus macerans JCM 2500t
Bacillus megaterium ATCC 13639
Bacillus megaterium IFO 12108
Bacillus megaterium JCM 2506t
Bacillus moritai ATCC 21282
Bacillus pabuli IFO 13638t
Paenibacillus polymyxa IFO 3020
Paenibacillus polymyxa JCM 2507t
Bacillus pumilus IFO 12092t
Bacillus sphaericus SC1713
Bacillus sphaericus ATCC 14577
Bacillus sphaericus IFO 3341
Bacillus sphaericus IFO 3525
Bacillus sphaericus IFO 3526
Bacillus sphaericus IFO 3527
Bacillus sphaericus IFO 3528
Bacillus subtilis JCM 1465t
Bacillus subtilis 168 NBRC111470
Bacillus subtilis ATCC 14593
Bacillus subtilis IFO 03108
Bacillus subtilis IFO 03134
Bacillus subtilis IFO 13169
Bacillus subtilis IFO 3026
Bacillus amyloliquefaciens NBRC3037
Bacillus thuringensis ATCC 13366
Bacillus validus IFO 13635

(Method)

The target bacterium-containing inorganic medium and the culture solution containing the bacterium to be evaluated were mixed by the same method as in Reference Experiment 4 to evaluate the microorganism degradation ability of the bacteria to be evaluated. The number of days that has elapsed on which the turbidity (OD660) of the target bacterium-containing inorganic medium showed 50% of the turbidity (OD660) of negative control was calculated and the presence or absence of microorganism degradation ability was decided by the following criteria.

A: 0 Days or more and less than 1.0 day

B: 1.0 Day or more and less than 4.0 days

C: 4.0 Days or more

Note that for the negative control, the turbidity (OD660) of target bacterium-containing inorganic medium to which the culture solution containing the bacterium to be evaluated was not added was used.

(Results)

The results are shown in Table 15. Further, a homology search of the nucleotide sequences of 16S rRNA genes of the bacteria to be evaluated shown in Table 14 against the nucleotide sequence (SEQ ID NO: 7) of 16S rRNA gene of Tumebacillus sp. NITE BP-02779 was carried out and calculated identity proportions are shown in Table 15. The nucleotide sequences of 16S rRNA genes of the bacteria shown in Table 15 are shown as SEQ ID NOs: 9 to 18. These are the sequences that are listed in National Institute of Technology and Evaluation, Biological Resource Center (NBRC) online catalogue
<https://www.nite.go.jp/nbrc/catalogue/NBRCDispSearchServlet?lang=jp> or National Center for Biotechnology Information, NCBI database catalogue
<https://www.ncbi.nlm.nih.gov/>.

Tumebacillus sp. NITE BP-02779 and Tumebacillus algifaecis NBRC108765t had Grade A but the Bacillus genus bacteria and the Paenibacillus genus bacteria had Grade B or Grade C. Thus, Tumebacillus sp. NITE BP-02779 and Tumebacillus algifaecis NBRC108765t had superior ability to degrade target microorganisms (target killed bacteria) to bacteria of the genus Bacillus and bacteria of the genus Paenibacillus.

The identity of the nucleotide sequence of 16S rRNA gene of Tumebacillus sp. NITE BP-02779 to the nucleotide sequence of 16S rRNA gene of Tumebacillus algifaecis NBRC108765t was 92.8%. On the other hand, the identity of the nucleotide sequence of 16S rRNA gene of Tumebacillus sp. NITE BP-02779 to the nucleotide sequence of 16S rRNA of the bacteria of the genus Bacillus or the bacteria of the genus Paenibacillus was less than 90%. This finding led to consider that the bacteria having 90% or more nucleotide sequence identity of 16S rRNA gene to Tumebacillus sp. NITE BP-02779 are superior in the ability to degrade target microorganisms. Tumebacillus algifaecis NBRC108765t can be obtained from NBRC, and the nucleotide sequence of 16S rRNA gene thereof is listed in GenBank/EMBL/DDBJ database under Accession No. JX110710.

TABLE 15

| | | Sequence |
|---|---|---|
| Bacteria to be evaluated | Results | identity |
| Tumebacillus sp. NITE BP-02779 | A | — |
| | (0.3 days) | |
| Tumebacillus algifaecis NBRC108765t | A | >90% |
| | (0.8 days) | (92.8) |
| Paenibacillus polymyxa JCM 2507t | B | <90% |
| | (1.1 days) | (85.3) |
| Bacillus amyloliquefaciens NBRC3037 | B | <90% |
| | (1.2 days) | (86.7) |
| Bacillus subtilis IFO 3134 | B | <90% |
| | (2.2 days) | (86.7) |
| Bacillus subtilis IFO 3026 | B | <90% |
| | (2.9 days) | (86.7) |
| Bacillus subtilis IFO 13169 | B | <90% |
| | (2.9 days) | (86.7) |
| Bacillus sphaericus IFO 3341 | B | <90% |
| | (3.3 days) | (86.8) |
| Bacillus sphaericus IFO 3528 | B | <90% |
| | (3.6 days) | (86.8) |
| Bacillus licheniformis IFO 12197 | B | <90% |
| | (3.6 days) | (87.0) |
| Bacillus subtilis 168 NBRC111470 | C | <90% |
| | (4.2 days) | (86.7) |
| Other (33 strains) | C | <90% |
| | (>4.2 days) | |

### Reference Experiment 9. Target Microorganism Degradation Ability Evaluation of a Mixed Solution Containing Tumebacillus Sp. NITE BP-02779 and Escherichia coli

(Materials)

For target microorganisms-containing inorganic medium, target bacteria (killed bacteria)-containing inorganic medium containing the bacteria of the genus Micrococcus as the target bacteria used in Reference Experiment 4 was prepared. Further, a solution containing Tumebacillus sp. NITE BP-02779 and Escherichia coli DH5α was prepared.

(Method)

First, Tumebacillus sp. NITE BP-02779 was cultured at 25° C. for 24 hours, and Escherichia coli DH5α was cultured at 37° C. for 24 hours.

After the culture, mixed solutions were prepared by mixing the Tumebacillus sp. NITE BP-02779 culture solution (prepared to be OD660=0.2) and the Escherichia co/i DH5α culture solution (prepared to be OD660=0.2) in ratios shown in Table 16. 50 μL of the prepared microbial preparation and 5.0 mL of the target bacterium-containing inorganic medium were added to a test tube, reacted at 25° C. and 200 rpm and measured for a turbidity (OD660) of the mixed solution 2 days later with an easy operation turbidity meter (easy operation OD monitor, miniphoto 518R, manufactured by TAITEC Corporation). A proportion of the turbidity (OD660) of the target bacterium-containing inorganic medium to which the microbial preparation was added to the turbidity (OD660) of the negative control was calculated as a survival rate of the target microorganism. The ability to degrade target microorganism was evaluated by the following criteria.

A: 0% or more and less than 50%

B: 50% or more and less than 70%

C: 70% or more

Note that for the negative control, the turbidity (OD660) of target bacterium-containing inorganic medium to which the mixed solution was not added was used.

(Results)

The results are shown in Table 16. The solution containing Tumebacillus sp. NITE BP-02779 and Escherichia coli demonstrated good ability to degrade target microorganisms.

TABLE 16

| | | Survival rate |
|---|---|---|
| | | of target |
| | | microorganisms |
| Microbial preparations | | (%) |
| Proportion of bacteria (a1) | 100% | A (5%) |
| to sum of bacteria (a1) and | 50% | A (22%) |
| microorganisms (a2) contained | 25% | A (28%) |
| in a microbial preparation | 13% | A (35%) |
| T/(T + E) × 100 | 6.3% | A (39%) |
| | 3.1% | A (36%) |
| | 1.6% | A (43%) |
| | 0.78% | A (42%) |
| | 0.39% | A (45%) |
| | 0.20% | A (48%) |
| | 0% | C (124%) |

| | |
|---|---|
| Negative control | C (100%) |

T: Number of Tumebacillus sp. NITE BP-02779
E: Number of Escherichia coli DH5α

Survival rate of target microorganism (%)=OD660 (microbial preparation added)/OD660 (negative control)×100

### Reference Experiment 10. Target Microorganism Degradation Ability Evaluation of Tumebacillus sp. NITE BP-02779 Culture

(Materials)

For a target bacterium (viable bacterium)-containing phosphoric acid buffer solution, a solution in which 10 mL of a 66 mM potassium phosphate buffer solution (pH 6.24) and 10 mg of dry bacterial cells of the bacteria of the genus Micrococcus (manufactured by SIGMA-ALDRICH) were mixed was used.

(Method)

First, Tumebacillus sp. NITE BP-02779 was cultured in R2A medium for a certain time.

After the culture, the culture solution was centrifuged, and the culture supernatant from which the bacterial cells were removed was collected. 100 μL of the collected culture supernatant and 100 μL of a target bacterium-containing phosphoric acid buffer solution were added to a 96-well plate and measured at ABS 450 nm over time using a microplate reader (manufactured by Molecular Device, LLC). The ability to degrade bacteria of the genus Micrococcus genus (UNITS/mL) was calculated and the ability to degrade target microorganisms was evaluated by the following criteria.

S: 200 (UNITS/mL) or more

A: 100 (UNITS/mL) or more and less than 200 (UNITS/mL)

B: 20 (UNITS/mL) or more and less than 100 (UNITS/mL)

C: Less than 20 (UNITS/mL)

Note that the ability to degrade bacteria of the genus Micrococcus (UNITS/mL) was calculated using the following expression.

Ability to degrade bacteria of the genus Micrococcus (UNITS/mL)={ΔABS 450 nm/min (target bacterium-containing phosphoric acid buffer solution to which culture supernatant was added)−ΔABS 450 nm/min (target bacterium-containing phosphoric acid buffer solution to which culture supernatant was not added)}/(0.001×0.1)

(Results)

The results are shown in Table 17. It was revealed that the culture supernatant of which Tumebacillus sp. NITE BP-02779 was cultured for a certain time had the ability to degrade target microorganisms.

TABLE 17

| Culture time | |
|---|---|
| (hr) | Micrococcus genus bacteria degradation ability |
| 0 | C (13 UNITS/mL) |
| 21 | A (121 UNITS/mL) |
| 28 | S (249 UNITS/mL) |
| 46 | S (250 UNITS/mL) |
| 53 | S (252 UNITS/mL) |
| 68 | A (153 UNITS/mL) |

1. A bacterium for degrading sludge having a 16S rRNA gene comprising a nucleotide sequence having 97% or more identity to the nucleotide sequence represented by SEQ ID NO: 1.

2. A bacterium having a 16S rRNA gene comprising the nucleotide sequence represented by SEQ ID NO: 1 with mutations of two bases or less, and having an ability to degrade a target microorganism.

3. The bacterium according to claim 2, wherein the target microorganism is a target bacterium.

4. The bacterium according to claim 2, wherein the target microorganism is a target killed bacterium.

5. The bacterium according to claim 1, having a 16S rRNA gene comprising the nucleotide sequence represented by SEQ ID NO: 1.

6. A bacterium deposited under Accession number NITE BP-03020.

7. A microbial preparation for degrading sludge, comprising a bacterium (a1):
bacterium (a1): a bacterium having a 16S rRNA gene comprising a nucleotide sequence having 90% or more identity to the nucleotide sequence represented by SEQ ID NO: 1.

8. The microbial preparation for degrading sludge according to claim 7, wherein the bacterium (a1) has a 16S rRNA gene comprising a nucleotide sequence having 95% or more identity to the nucleotide sequence represented by SEQ ID NO: 1.

9. The microbial preparation for degrading sludge according to claim 7, wherein the bacterium (a1) has a 16S rRNA gene comprising a nucleotide sequence having 97% or more identity to the nucleotide sequence represented by SEQ ID NO: 1.

10. The microbial preparation for degrading sludge according to claim 7, wherein the bacterium (a1) has a 16S rRNA gene comprising the nucleotide sequence represented by SEQ ID NO: 1.

11. The microbial preparation for degrading sludge according to claim 7, wherein the bacterium (a1) has an ability to degrade a target microorganism.

12. The microbial preparation for degrading sludge according to claim 7, which degrades sludge at a temperature of 20° C.

13. The microbial preparation for degrading sludge according to claim 7, wherein the bacterium (a1) is a bacterium deposited under Accession number NITE BP-03020.

14. The microbial preparation for degrading sludge according to claim 7, further comprising a microorganism (a2):
microorganism (a2): a microorganism different from bacterium (a1), wherein the percentage of the bacteria (a1) based on the total number of the bacteria (a1) and the microorganism (a2) is 0.1% or more and less than 100%.

15. The microbial preparation for degrading sludge according to claim 14, wherein the microorganism (a2) has a 16S rRNA gene comprising a nucleotide sequence having 90% or more identity to the nucleotide sequence represented by SEQ ID NO: 7, and comprises at least one species selected from the group consisting of bacteria having an ability to degrade a target microorganism, bacteria of the genus Bacillus having an ability to degrade a target microorganism, and bacteria of the genus Paenibacillus having an ability to degrade a target microorganism.

16. A microbial preparation for degrading sludge, comprising a culture of a bacterium (a1):
bacterium (a1): a bacterium having a 16S rRNA gene comprising a nucleotide sequence having 90% or more identity to the nucleotide sequence represented by SEQ ID NO: 1, and having an ability to degrade a target microorganism.

17. A method for degrading sludge, comprising allowing the bacterium according to claim 1 to act on sludge.

18. A device for degrading sludge, wherein the bacterium according to claim 1 is used.

19. A method for degrading sludge, comprising allowing the microbial preparation for degrading sludge according to claim 7 to act on sludge.

20. A device for degrading sludge, wherein the microbial preparation for degrading sludge according to claim 7 is used.
